SEQUENCE LISTING (5 sequences)

SEQ ID NO: 1 - DNA, 860 bp, Thermus brockianus
  CDS (3)..(860): Catalase coding sequence
tagaccgcctgcagattgagttgcccatgcccaaggagcaggaccccaacgccgccgccgcggtgcaggcccttctgggcggtcgcttcggggagatgtccaccctgatgaactacatgtaccagtccttcaacttccgggggaagaaggcgcttaagccctactacgacctcatcgccaacatcgccacggaggagcttgggcacattgagctcgtttccgccaccatcaacagcctcctggccaaaaaccccggaaaggacctggaggaaggggtagaccccgtgagcgcccccttgggcttctccaaggacgcccgcaacgccgcccacttcatcgccgggggggccaacaccctggtgatgggggccatgggagagcactggcacggggagtacgtcttcaccagcggcaacctcatcctggaccttctccacaacttcttcctggaggtggcggcccgcacccacaagctccgggtctacgagatgacggataaccccgtggcccgggagatgatcggctacctcctggtgcggggtggggtccacgccgccgcctacggcaaggccctggaaacccttaccggggtggagatgaccaagatgctccccatcccccggattgacaacagcaagatccccgaggccaagaagtacatggacctgggcttccaccgcaacctctaccgctttagcccctccgattaccaggacctgggccttatctggaatggggcttcccccgaggacgggagcgaggtggtggtggtggacggcccccccacgggcggccccgtgtttgacgccggccacgacgccgccgagttcgcccccgagttccaccccgccgagctctac

SEQ ID NO: 2 - protein, 286 aa, Thermus brockianus
DRLQIELPMPKEQDPNAAAAVQALLGGRFGEMSTLMNYMYQSFNFRGKKALKPYYDLIANIATEELGHIELVSATINSLLAKNPGKDLEEGVDPVSAPLGFSKDARNAAHFIAGGANTLVMGAMGEHWHGEYVFTSGNLILDLLHNFFLEVAARTHKLRVYEMTDNPVAREMIGYLLVRGGVHAAAYGKALETLTGVEMTKMLPIPRIDNSKIPEAKKYMDLGFHRNLYRFSPSDYQDLGLIWNGASPEDGSEVVVVDGPPTGGPVFDAGHDAAEFAPEFHPAELY

SEQ ID NO: 3 - protein, 23 aa, Thermus brockianus
MFLRIDRLQIELPMPKEQDPNAA

SEQ ID NO: 4 - protein, 17 aa, Thermus brockianus
MFLRIDRLQIELPPPPE

SEQ ID NO: 5 - protein, 302 aa, Thermus brockianus
  MISC_FEATURE (14)..(14): Xaa may be Pro or Met
MFLRIDRLQIELPXPXEQDPNAAAAVQALLGGRFGEMSTLMNYMYQSFNFRGKKALKPYYDLIANIATEELGHIELVSATINSLLAKNPGKDLEEGVDPVSAPLGFSKDARNAAHFIAGGANTLVMGAMGEHWHGEYVFTSGNLILDLLHNFFLEVAARTHKLRVYEMTDNPVAREMIGYLLVRGGVHAAAYGKALETLTGVEMTKMLPIPRIDNSKIPEAKKYMDLGFHRNLYRFSPSDYQDLGLIWNGASPEDGSEVVVVDGPPTGGPVFDAGHDAAEFAPEFHPAELYXIAKKLYEKAK

(19) United States
(12) Patent Application Publication
(10) Pub. No.: US 2005/0112742 A1
(43) Pub. Date: May 26, 2005

(54) High temperature and alkaline stable catalase

(76) Inventors: Vicki Thompson, Idaho Falls, ID (US); William A. Apel, Idaho Falls, ID (US); Kastli D. Schaller, Pocatello, ID (US)

Correspondence Address: Stephen R. Christian, P.O. Box 1625, Idaho Falls, ID, 83415-3899, US

(21) Appl. No.: 10/981,434

(22) Filed: Nov. 3, 2004

Related U.S. Application Data
(60) Provisional application No. 60/517,976, filed on Nov. 5, 2003.

(51) Int. Cl. C12P 3/00; C12N 9/08

(52) U.S. Cl. 435/168; 435/192

(57) ABSTRACT

The invention relates to thermal and pH stable catalases. One catalase of the invention was purified and characterized from Thermus brockianus. As a part of the characterization, the enzyme was compared to typical catalases from commercial sources and found to be significantly more thermal/alkaline stable than these other enzymes. The catalase purified from T. brockianus consists of four identical subunits having a molecular mass of approximately 42.5 kDa, for a total molecular mass of approximately 178 kDa.

### CROSS-REFERENCE TO RELATED APPLICATIONS

This application claims the benefit of U.S. Provisional Patent Application No. 60/517,976, filed Nov. 5, 2003.

### GOVERNMENT RIGHTS

The United States Government has rights in the following invention pursuant to Contact No. DE-AC07-99ID13727 between the U.S. Department of Energy and Bechtel BWXT Idaho, LLC.

### BACKGROUND OF THE INVENTION

1. Field of the Invention

The invention relates to a thermally stable catalase and methods of using a thermally stable catalase.

2. State of the Art

In general, a catalase (EC 1.11.1.6) is an enzyme that catalyzes the decomposition of hydrogen peroxide to oxygen and water. Cells utilize catalases, together with other cellular enzyme systems, to protect themselves against the harmful effects of reactive oxygen species such as super-oxide anions, hydrogen peroxide, and hydroxyl radicals.

In recent years there has been growing interest in utilizing hydrogen peroxide in industrial sectors as a more environmentally friendly alternative to existing chemical treatments for bleaching and sterilization. For example, the Scientific Committee On Toxicity, Ecotoxicity And The Environment (CSTEE) has reported the usage of approximately 670,000 tons of hydrogen peroxide in the European Union (EU). This usage includes pulp bleaching (48%) and as an intermediate in the synthesis of other substances (38%), textile bleaching (7%), water treatment (3%) and other miscellaneous uses (5%). Id.

A driving force behind the increased use of hydrogen peroxide relates to its reduced environmental impact and reduced hazard relative to an equivalent amount of chlorine. Nevertheless, the Scientific Committee On Toxicity has concluded that environmental exposure to hydrogen peroxide may occur through emissions in all major environmental compartments, air, surface water, and soil. Id. Thus, there is a need to treat hydrogen peroxide to reduce environmental exposure. Furthermore, the use of hydrogen peroxide, for example, in industrial settings, frequently requires that it be removed from the process stream, since it can interfere with subsequent process steps. Thus, there is a need to remove hydrogen peroxide from the process stream so that the process water may be reused in subsequent steps.

The catalase enzyme has been used in the textile industry as a milder, more environmentally conscious method of removing or decreasing residual hydrogen peroxide in exhausted bleach baths. However, bleaching of textiles, pulp and paper typically occurs at high temperatures and pH. At these elevated temperatures and pH, commercially available catalases do not retain sufficient activity to provide an economically practical method of removing the hydrogen peroxide. Thus, the temperature and pH of the process water must be reduced prior to treatment with traditional catalases.

In particular, the bleaching of fabrics in the textile industry provides one example of hydrogen peroxide use where removal of the hydrogen peroxide from the process stream, subsequent to its intended use therein, would be beneficial, since it has been shown that hydrogen peroxide interferes with the subsequent dying steps.

Current methods to remove hydrogen peroxide either utilize extensive washing, which results in the generation of large volumes of wastewater, or utilize chemical treatments such as sodium bisulfite or hydrosulfite to reduce hydrogen peroxide, which leads to high salt levels in the process stream. Although catalases have been tried as a solution to the above problem, the lack of stability limits their large scale use. Specifically, the use of a non-thermally tolerant catalase to remove excess hydrogen peroxide is problematic, since many industrial processes utilizing hydrogen peroxide occur at elevated temperatures and pH (>60° C. and pH 9). Thus, the currently available commercial enzymes, which rapidly lose their activity under these conditions, are unsuitable for use under such conditions. For example, to utilize non-thermo tolerant enzymes in an industrial process operating at an elevated temperature, either the temperature must be adjusted downwardly prior to addition of the catalase or the enzyme has to be continually replenished as it loses activity. Furthermore, the temperature of the process stream may have to be raised again following treatment with a non-thermostable catalase. Thus, the process of modifying the temperature to accommodate a non-thermo tolerant catalase or continually replenishing the catalase represents an economic inefficiency of non-thermo tolerant catalases, which can be overcome through the use of a thermostable catalase.

Enzymes, such as catalases, are proteins and undergo increased denaturation (i.e., a conformational alteration resulting in the loss of biological activity) at elevated temperatures. Generally, the rate of denaturation, or more generally, the rate of deactivation, increases in a non-linear fashion as the temperature increases. Thus, the actual deactivation of the catalase is a product of the deactivation rate and the duration of incubation.

If deactivation were the only factor influencing optimal enzyme use parameters, lower temperatures would be preferable. However, inactivation by heat must be balanced with a temperature-dependent increase in the enzymatic rate of catalysis that accompanies increasing temperature, up to an optimum temperature, which is often a temperature where deactivation of the enzyme is of concern. Thus, temperature plays a significant role in enzyme performance.

In addition to temperature, pH also affects enzyme kinetics and stability of the enzyme. The pH may affect deactivation of the enzyme due to covalent changes, such as the deamination of asparagine residues and non-covalent changes such as the rearrangement of the protein chain. High pH, indicative of a basic or alkaline environment, may also result in random cleavage of the peptide. Beyond deamination and cleavage, pH has a substantial effect on the protonation state of the amino acid side chains and the function of the enzyme. Thus, enzymes display a range of pH within which they will function adequately. In particular, commercially available catalases are optimally active at a temperature range between 20-50° C. and at neutral pH.

Three general classes of catalases have been described in the literature: the typical or monofunctional catalases; the catalase-peroxidases that have a peroxidative activity as well as catalase activity; and the Mn-catalases or pseudocatalases. Typical catalases, which have similar properties, have been isolated from numerous animals, plants, and microorganisms. These enzymes typically have four subunits of equal size with a combined molecular mass of 225,000-270,000 kDa and characteristically have four protoheme IX prosthetic groups per tetrameric molecule. These enzymes also typically display a broad pH activity range from 4 to 10, are specifically inhibited by 3-amino-1,2,4-triazole, and are resistant to reduction by dithionite.

Most of the reported catalases utilize protoheme IX. Although, there are a few reports of other types of hemes such as heme d in the HPII catalase from E. coli, a novel heme type in the catalase from N. crassa, and heme b in a catalase-peroxidase from Synechocystis PCC 6803. Crystal structures solved for catalases from a variety of organisms indicate that the heme iron is 5-coordinate in the native resting state with positions 1-4 occupied by the four pyrrole nitrogens of the heme group, position 5 on the proximal side of the heme occupied with the amino acid tyrosine, and the 6 position on the distal side of the heme vacant. The distal side of the heme is where the catalatic reaction is proposed to occur in these catalases. In the resting state, the absence of a ligand in the 6 position allows the electrons of the iron to be unpaired, resulting in a high spin state. In the presence of a ligand such as cyanide, the heme iron becomes 6-coordinate with a strong ligand field resulting in only one unpaired electron in the heme iron and a corresponding low spin state.

A catalase-peroxidase enzyme was first isolated from Escherichia coli in 1979. These enzymes are typically dimers or tetramers with a subunit size of approximately 80 kDa and, in contrast to the typical catalases, generally have a low heme content with only 1-2 hemes per enzyme molecule. Additionally, the catalase-peroxidases typically have a sharp pH optimum, are not inhibited by 3-amino-1,2,4-triazole, are sensitive to hydrogen peroxide concentration, and are readily reduced by dithionite. Sequence analysis of the two groups of enzymes has shown that they are not related and on the basis of sequence similarity, the catalase-peroxidases are grouped in class I of the superfamily of plant, fungal, and bacterial peroxidases. Both catalase and catalase-peroxidases are strongly inhibited by cyanide and azide, both of which are classic heme protein inhibitors.

The Mn-catalases, in contrast to the other two catalase groups, do not utilize a heme prosthetic group in their active site and, instead, use manganese ions. Therefore, these enzymes should be insensitive to the heme poisons, cyanide, and azide. The Mn-catalases, or pseudocatalases, typically have subunit sizes ranging from 28 to 35 kDa and are hexameric. However, a tetrameric pseudocatalase enzyme was described from Thermoleophilum album (Allgood and Perry, Characterization of a manganese-containing catalase from the obligate thermophile Thermoleophilum album. J. Bacteriol. (1986), 168(2):563-567).

The few thermostable versions of a monofunctional catalase (Wang et al., Purification and characterization of a thermostable catalase from culture broth of Thermoascus aurantiacus. J. Ferment. Bioeng. (1998), 85(2):169-173), catalase-peroxidases (Kengen et al., Characterization of a catalase-peroxidase from the hyperthermophilic archaeon Archaeoglobus fulgidus. Extremophiles (2001), 5:323-332; Apitz and van Pee, Isolation and characterization of a thermostable intracellular enzyme with peroxidase activity from Bacillus sphaericus. Arch. Microbiol. (2001), 175:405-412; Gudelj, et al., A catalase-peroxidase from a newly isolated thermoalkaliphilic Bacillus sp. with potential for the treatment of textile bleaching effluents. Extremophiles (2001), 5:423-429; and Loprasert, et al. Thermostable peroxidase from Bacillus stearothermophilus. J. Gen. Microbiol. (1988), 134:1971-1976), or Mn-catalases (Allgood and Perry, Characterization of a manganese-containing catalase from the obligate thermophile Thermoleophilum album. J. Bacteriol. (1986), 168(2):563-567; and Kagawa et al., Purification and cloning of a thermostable manganese catalase from a thermophilic bacterium. Arch. Biochem. Biophys. (1999), 363(2):346-355) that have been described do not possess the desirable properties of the invention. Many of these reported enzymes exhibited low thermal stability at temperatures above 60° C., several were rapidly inactivated in the presence of hydrogen peroxide, and most of the enzymes had low activity and stability at elevated temperature and pH, making them unsuitable for many applications.

In particular, Mn-catalases have been isolated from three thermophilic organisms: Thermus species strain YS 8-13, Thermus thermophilus, and Thermoleophilum album. These catalases were reported to be thermostable and pH stable, but stability was only examined over the course of a few hours. No studies were done examining stability at both high temperature and high pH. Catalase-peroxidases have been found in several thermophilic organisms: Archaeoglobus fulgidus, Bacillus stearothermophilus, and Bacillus sp. SF. These enzymes were also reported to be thermostable, but no long term studies were conducted. The Bacillus SF catalase-peroxidase was much less stable at high temperature and pH than a catalase of the invention. These other enzymes also lacked stability in the presence of hydrogen peroxide.

In addition, a heme catalase was purified from Thermoascus aurantiacus, which is reported to have activity over the range of 30-90° C. with optimum activity at 70° C.; however, at 85° C. this enzyme had only 20% of its initial activity after 8 hours of incubation, and retained only 40% of initial activity when incubated at a pH of 10. A Mn-catalase from T. album has a reported activity over the range of 25-60° C. with an optimum temperature for activity at 35° C. In addition, this Mn-catalase lost 10% of its activity after 1 h of incubation at 80° C. and 7% of its activity after 24 h of incubation at 60° C. A Mn-catalase, isolated from Thermus sp., is reported to have a maximum activity at 85° C. and to be active over a temperature range from 40 to 90° C. The thermo-alkali-stable catalase purified from Bacillus sp. SF for potential treatment of textile bleaching effluents had half-lives of only 38 and 4 h when incubated at pH 9 and 10 and 60° C., respectively.

In contrast to the present invention, commercially available catalases exhibit little to no activity under conditions of elevated temperature and high pH.

### BRIEF SUMMARY OF THE INVENTION

The invention relates to a thermostable catalase protein from the genus Thermus, nucleic acid sequences encoding the catalase, and to a method for using the nucleic acid or protein sequences for catalyzing the conversion of hydrogen peroxide to water and oxygen.

In one exemplary embodiment, the invention relates to a catalase having an activity half-life of at least about 200 hours at a temperature of about 80° C. and a pH of about 8.0 and that demonstrates substantially no substrate inhibition at hydrogen peroxide concentrations up to about 450 mM. An exemplary catalase was obtained from T. brockianus.

In a further exemplary embodiment, the invention also relates to an isolated thermostable catalase produced by the process of: growing a microorganism having catalase activity; preparing a cell lysate from the microorganism; identifying a catalase activity in the cell lysate; purifying the catalase activity from the cell lysate; demonstrating the absence of substantial substrate inhibition of the catalase activity at a hydrogen peroxide concentration between about 200 and about 450 mM; and determining a half-life for the catalase, wherein the catalase has a half-life of at least about 200 hours at a temperature of about 80° C. and a pH of about 8.0.

The invention also relates to a method of purifying the catalase, which includes chromatographing a cell extract using at least one of an ion-exchange column, a hydrophobic interaction column and a gel filtration column to produce a purified catalase.

In another exemplary embodiment, the catalase, for example, the catalase purified by chromatographing a cell extract according to the invention, may also have a pyridine hemochrome spectra indicative of heme c.

In an additional exemplary embodiment, the invention relates to a method of converting hydrogen peroxide to oxygen and water under conditions of high temperature and pH, comprising: adding a sample containing hydrogen peroxide to a catalase; incubating the catalase with the hydrogen peroxide solution at a high temperature and at an alkaline pH; and converting a desired amount of the hydrogen peroxide to oxygen and water. The term “high temperature” includes temperatures between about 70° C. and about 90° C. An alkaline pH includes pH values between about 8 and about 10 or any range between about 8 and about 10, for example, between about 8.5 and about 9.5. The method may be used to treat a sample containing hydrogen peroxide that is obtained from bleaching pulp, paper or textile. Furthermore, the method may be used in combination with an immobilized catalase. For example, the sample may be passed through a column of immobilized catalase.

The invention further relates to an isolated nucleic acid comprising a nucleic acid sequence encoding a polypeptide having the sequence set forth in SEQ ID NO:2 or SEQ ID NO:5, a nucleic acid sequence encoding a polypeptide having between about 75 and about 95% or between about 85 and about 99% identity to the sequence set forth in SEQ ID NO:2, SEQ ID NO:5 or a functional fragment thereof. The nucleic acid may be present in a vector, an expression vector, and/or a host cell.

The invention also relates to an isolated catalase comprising a polypeptide having the sequence set forth in SEQ ID NO:2 or SEQ ID NO:5, a polypeptide having between about 75 and about 95% or between about 85 and about 99% identity to the sequence set forth in SEQ ID NO:2, SEQ ID NO:5, or a functional fragment thereof. The polypeptide may be produced by chemical synthesis or produced in vivo or in vitro. The invention also relates to an isolated polypeptide having about 95%, about 96%, about 97%, about 98%, and about 99% identity to the sequence set forth in SEQ ID NO:2, SEQ ID NO:5, or a functional fragment thereof, and/or a nucleic acid encoding the polypeptide. The invention includes individually and/or in combination each amino acid sequence encompassed by SEQ ID NO:5, for example, the amino acid sequence wherein the first Xaa position of SEQ ID NO:5 is methionine, the second amino acid position is lysine, and the third Xaa position (not shown above) is either amino acid and all other combinations.

The invention also relates to functional fragments of the catalase. The catalase of the invention includes fragments of the catalase wherein catalase activity is retained. For example, amino acid substitutions and deletions outside of the heme or Mn binding pocket, wherein the protein retains catalase activity, are included in one aspect of the invention. Further, deletions and truncations of the polypeptide, which retain enzymatic activity, may be made by a person of ordinary skill in the art and fall within the scope of the invention.

The invention also relates to a host cell containing a nucleic acid encoding a thermal tolerant catalase. For example, the host cell may be used to express the catalase and may be used as a means of producing the catalase.

The invention relates to the attachment of a catalase to a water-insoluble solid support (immobilization), as well as to an immobilized catalase and analytical tools in the form of biosensors, which may incorporate an immobilized catalase. One aspect of this embodiment allows process water to be passed through or over an immobilized catalase, wherein the catalase converts hydrogen peroxide to oxygen and water without producing undesirable byproducts or contributing the catalase to the process water. Another aspect of the invention relates to increased stability due to immobilization, which can increase the temperatures and pH at which the catalase may be used.

### BRIEF DESCRIPTION OF THE SEVERAL VIEWS OF THE DRAWINGS

FIG. 1 is an SDS-PAGE of catalase containing fractions after each purification step: (Lane 1) molecular mass standards; (Lane 2) cellular extract; (Lane 3) DEAE ion exchange; (Lane 4) hydrophobic interaction; (Lane 5) gel filtration; and (Lane 6) molecular mass standards.

FIG. 2 represents the catalase activity as a function of temperature. Error bars represent one standard deviation from triplicate measurements.

FIG. 3 represents the catalase activity as a function of pH. Error bars represent one standard deviation from triplicate measurements.

FIG. 4 represents the temperature stability of the T. brockianus catalase enzyme incubated at (a) 80° C. or (b) 90° C.; and (c) initial activation of catalase activity at 80° C. Error bars represent one standard deviation from triplicate measurements.

FIG. 5 represents the rate of hydrogen peroxide decomposition as a function of hydrogen peroxide concentration. The solid line represents a nonlinear fit of Vₘ and Kₘₐₓ to the Michaelis-Menton equation. Error bars represent one standard deviation from triplicate measurements.

FIG. 6 is the absorption spectra of native enzyme, enzyme treated with 1 mM sodium dithionite, and enzyme treated with 10 mM KCN.

FIG. 7 is the pyridine hemochrome absorption spectrum of the T. brockianus catalase.

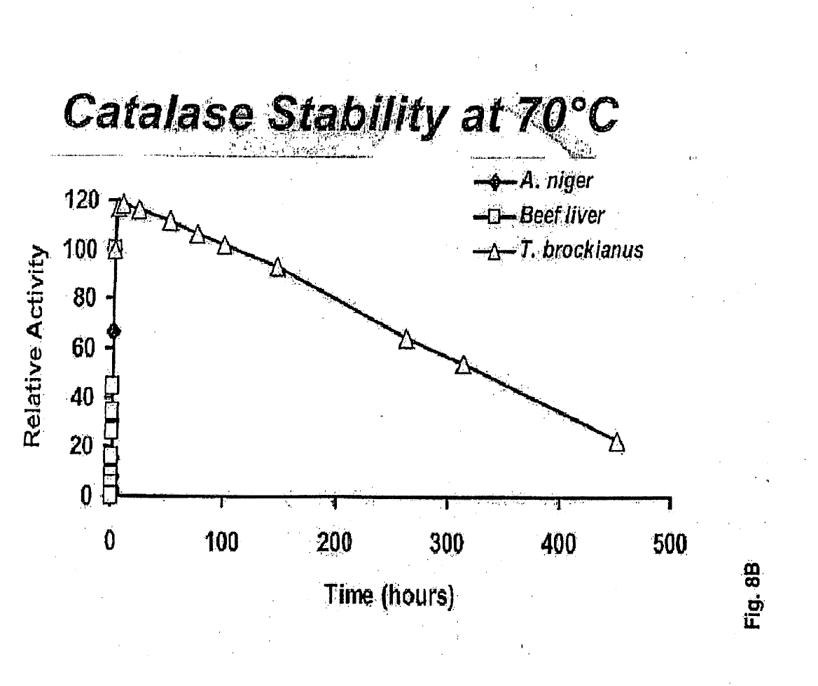

FIGS. 8A and B represent the catalase stability at 70° C. for catalases from A. niger (♦), beef liver (□) and T. brockianus (Δ).

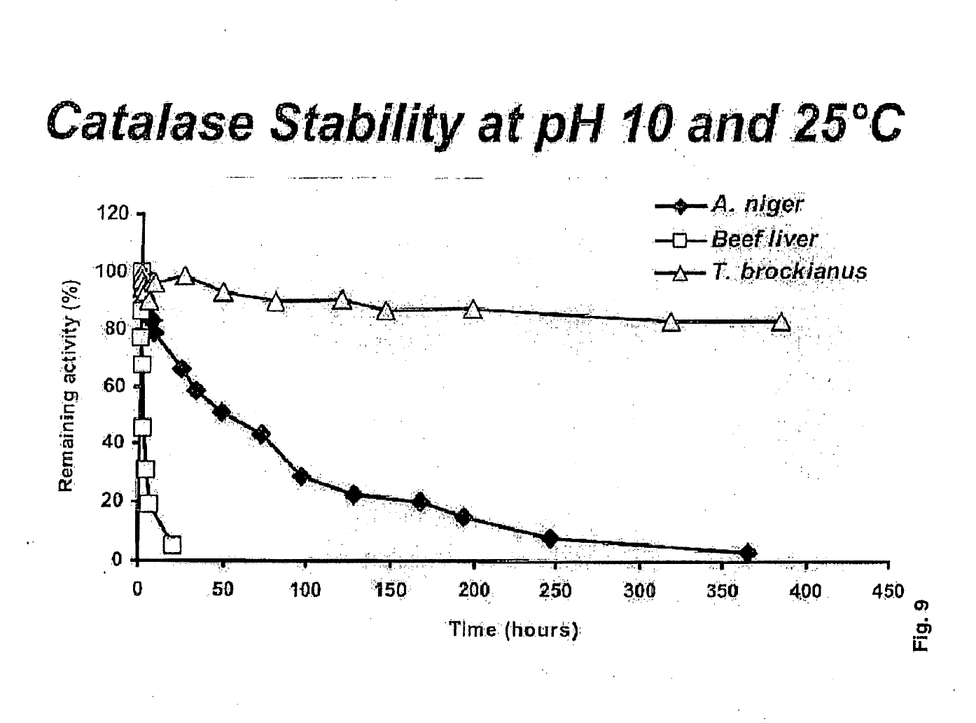

FIG. 9 represents the catalase stability at a pH of 10 and a temperature of 25° C. for catalases from A. niger (♦), beef liver (□) and T. brockianus (Δ).

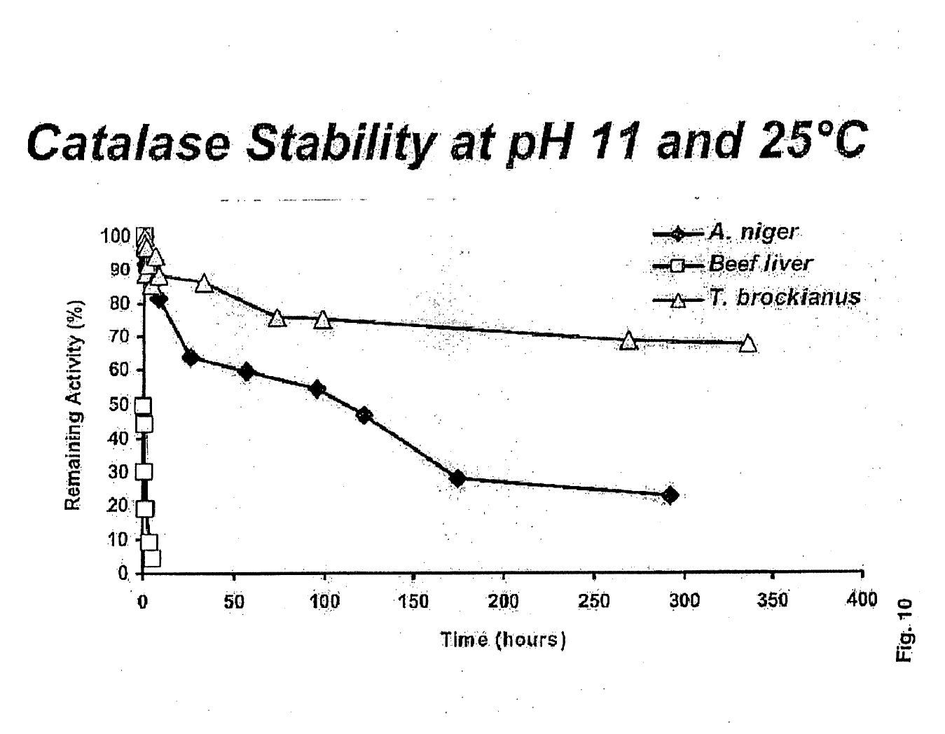

FIG. 10 represents the catalase stability at a pH of 11 and a temperature of 25° C. for catalases from A. niger (♦), beef liver (□) and T. brockianus (Δ).

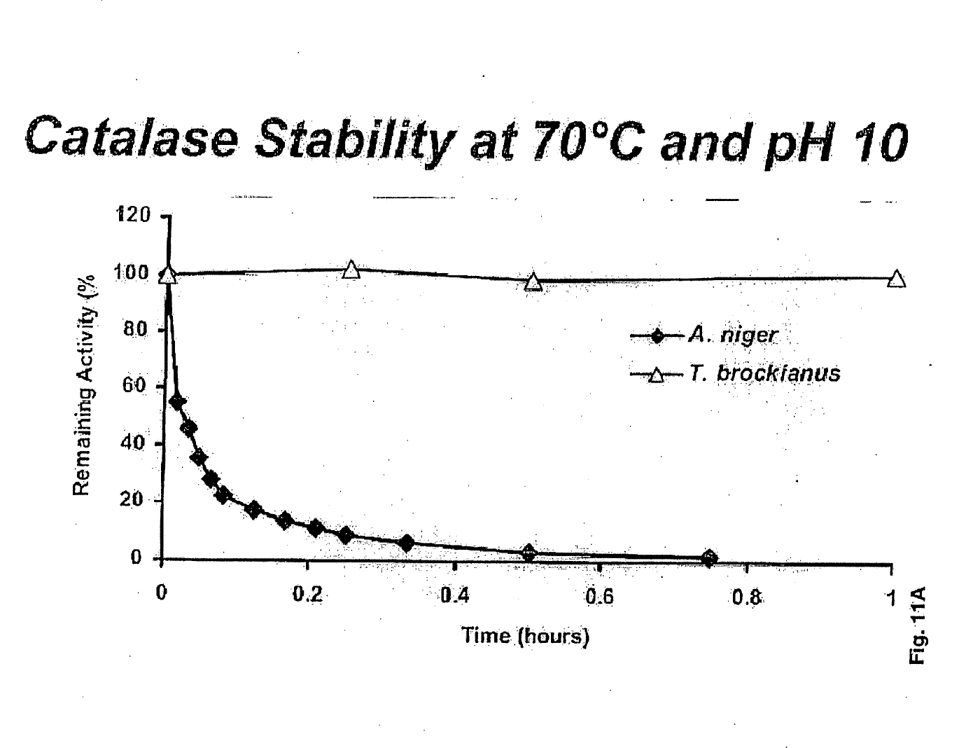

FIGS. 11A and B represent the catalase stability at a pH of 10 and a temperature of 70° C. for catalases from A. niger (♦), beef liver (□) and T. brockianus (Δ).

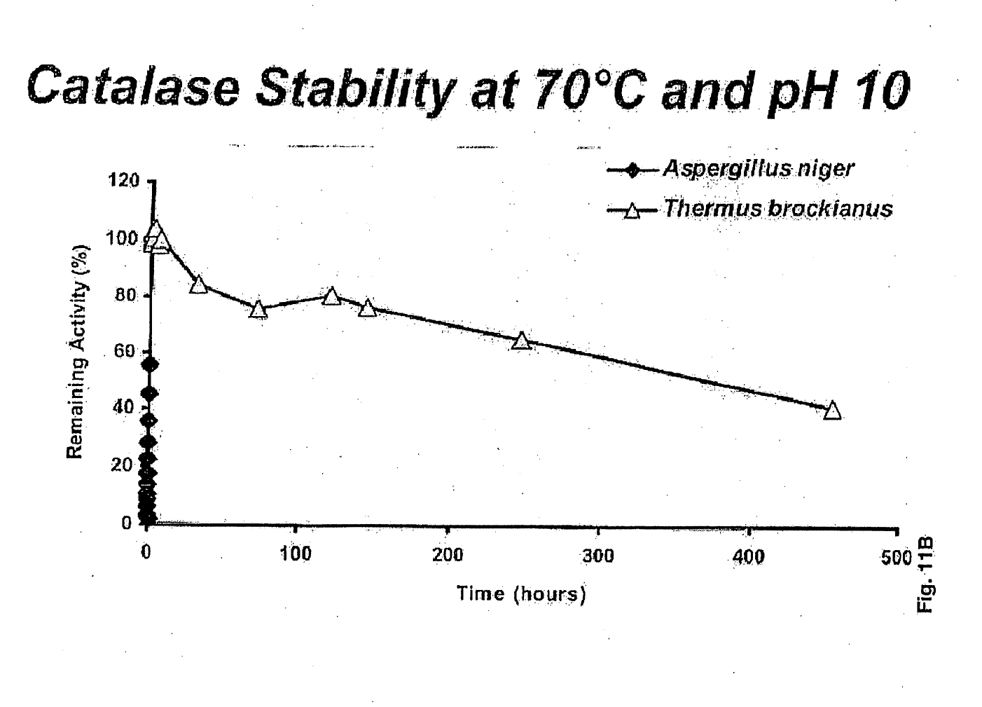

### DETAILED DESCRIPTION OF THE INVENTION

A catalase of the invention was purified and characterized from Thermus brockianus. As a part of the characterization, the enzyme was compared to typical catalases from commercial sources and found to be significantly more thermal/alkaline stable than these other enzymes. The catalase purified from T. brockianus comprises four identical subunits having a molecular mass of approximately 42.5 kDa, for a total molecular mass of approximately 178 kDa. The catalase was active from about 30-94° C. and a pH range from about 6-10. Optimum activity occurred at about 90° C. and about a pH of 8. At a pH of 8, the enzyme was extremely stable with half-lives of 330 hours at 80° C. and 3 hours at 90° C. The enzyme also demonstrates excellent stability at 70° C. and alkaline pH with measured half-lives of 510 hours and 360 hours at pHs of 9 and 10, respectively. By comparison, the catalase from the fungus Aspergillus niger has half-lives of 30 seconds and 15 seconds at 70° C. and a pH of 9 and 10, respectively. The half-life (tₕ) may be calculated using the following formula: tₕ=(ln2/k_{d}). Where k_{d} is the deactivation rate constant, which can be obtained from V=V₀e^{-k}d^{t}, where V₀ is the initial enzyme activity.

In addition, a Km of 35.5 mM and a Vmax of 20.3 mM/min·mg protein for hydrogen peroxide was measured for the catalase and the enzyme was not inhibited by hydrogen peroxide at concentrations up to about 450 mM.

The analysis of the absorption spectra for the catalase preparation indicates that the catalase may have an unusual heme active site utilizing 8 molecules of heme c per tetramer, rather than the protoheme IX present in the majority of catalases. This analysis also indicates that the heme iron of the catalase may exist in a 6-coordinate low spin state, rather than the typical 5-coordinate high spin state associated with other catalases.

The above properties indicate that the catalase purified from T. brockianus can function in high temperature and pH settings, for example, the industrial bleaching process where the catalase may be used to remove residual hydrogen peroxide from the process stream without requiring a decrease in temperature or pH. In particular, the process stream in an industrial bleaching process typically has a temperature of 60° C. or higher and pHs ranging from 9-11.

The term “purified” as used herein, is intended to refer to a nucleic acid or polypeptide, isolatable from other components, wherein the nucleic acid or polypeptide is purified to any degree relative to other components associated with the natural form. Generally, “purified” will refer to nucleic acid or polypeptide that has had one or more other components removed, and wherein a polypeptide substantially retains its expressed biological activity.

Various methods for quantifying the degree of purification of a nucleic acid or polypeptide will be known to those of skill in the art in light of the present disclosure. These methods include, but are not limited to, determining the absorption of a sample at an appropriate wavelength, determining the specific activity of a sample, determining the purity by chromatograph, for example, HPLC, or assessing the amount of a polypeptide within a sample by SDS/PAGE analysis. A preferred method for assessing the purity of a sample containing a polypeptide is to calculate the specific activity of the sample, to compare it to the specific activity of the initial extract, and to thus calculate the degree of purity. The actual units used to represent the amount of activity will, of course, be dependent upon the particular assay technique chosen to follow the purification and the nature of the activity.

In an exemplary embodiment, the invention is used to convert hydrogen peroxide to oxygen and water without the production of undesirable byproducts. Thus, the invention may be used to treat hydrogen peroxide containing solutions.

In particular, the invention may be used where the conditions for catalysis are at a high temperature, a high pH, a high concentration of hydrogen peroxide, or a combination thereof. A high temperature includes temperatures between about 60 and about 100° C. and, more particularly, between about 70 and about 90° C. An alkaline or high pH includes a pH between about 7.5 and about 11, and between about 8 and about 10.

Generally, in processes where hydrogen peroxide is present, the invention may be used to reduce, remove or detect hydrogen peroxide, for example, in the production of glyoxylic acid and in glucose sensors. Also, in processes where hydrogen peroxide is used as a bleaching or antibacterial agent, the catalase may be used to remove or reduce residual hydrogen peroxide, for example, in contact lens cleaning, in bleaching steps in pulp and paper preparation, semiconductor manufacture, and in pasteurization, such as, pasteurization of dairy products. Further, the catalase may be used as a catalyst for oxidation reactions, e.g., epoxidation and hydroxylation.

Pulp bleaching and brightening with hydrogen peroxide is commonly used in the pulp and paper industry. Thus, in one exemplary embodiment, the invention may be used to remove hydrogen peroxide following brightening or bleaching. In another exemplary embodiment, the invention may be used to remove or reduce the concentration of H₂O₂ for environmental (pollution control/clean-up) purposes. The invention may also be used to reduce or remove hydrogen peroxide used to dye hair, fur or synthetic fibers. In a further exemplary embodiment, the invention may be used to remove or reduce hydrogen peroxide in semiconductor fabrication process. In still another exemplary embodiment of the invention, the catalase may be used to generate or supply oxygen by treating hydrogen peroxide in the presence of the catalase.

In an additional exemplary embodiment, the invention may be used in the production of textiles. For example, during weaving, the warp (chain) threads are exposed to considerable mechanical strain, and to prevent breaking, are usually reinforced by coating (sizing) with a gelatinous substance (size). As a consequence of the sizing, the warp threads of the fabric are not able to absorb water or finishing agents to a sufficient degree. Thus, the size must generally be removed (desizing) before finishing. In most cases, chemical breakdown of the size polymer in a separate desizing treatment is necessary in order to obtain the desired quality of the final fabric. In a conventional process of desizing, the breakdown of the size polymer is carried out using oxidizing agents such as ammonium persulfate or hydrogen peroxide at high pH and temperature. Thus, the invention may be used to remove the hydrogen peroxide following desizing.

In an exemplary embodiment, the invention may be used to remove or reduce the hydrogen peroxide content of an exhausted bleach bath, such as a bleach bath used in the bleaching of fabric, pulp or paper. In particular, textile production frequently requires bleaching of the starting material, in order to produce a product, such as a textile, having a sufficiently pure white color. Oxidative bleaches are frequently used in a process which is believed to oxidize the color bodies in the natural material into colorless compounds. Bleaching with chemicals such as hypochlorite have been known and used in the art, but the chlorinated byproducts are undesirable. Thus, the major bleaching agents currently used in textile, pulp and paper preparation are sodium hypochlorite, hydrogen peroxide and sodium chlorite. It is estimated that, today, 90 to 95% of all cotton and cotton/synthetic blends are bleached with hydrogen peroxide. In addition to interference with subsequent process steps, hydrogen peroxide is a corrosive, oxidizing agent which may cause combustion when allowed to dry out on oxidizable organic matter. Hydrogen peroxide is also an irritant to the skin and mucous membranes and dangerous to the eyes.

Hydrogen peroxide is an extremely weak acid; Kₐ=2.4×10¹² with a pKa of about 11.62. Since the perhydroxyl ion is the desired bleaching species, the pH may be adjusted to provide an optimum concentration of perhydroxyl ion. At a pH>11, there is a rapid generation of perhydroxyl ions and when the pH reaches about 11.8, all of the hydrogen peroxide is converted to perhydroxyl ions and bleaching is said to be out of control. The hydrogen peroxide anion concentration can be evaluated by a person of ordinary skill in the art using known equations and methods. Since stabilized hydrogen peroxide does not decompose at high temperature, the bleaching process may be conducted at a temperature of up to about 95° C. to about 100° C. Thus, the present invention may be used to remove hydrogen peroxide from bleach water.

The invention also relates to the amino acid sequence of a T. brockianus catalase or an allelic variant thereof. In an exemplary embodiment, the invention relates to the amino acid sequence of the T. brockianus catalase as set forth in SEQ ID NO:2, SEQ ID NO:5, or an allelic variant thereof. In an embodiment, the invention relates to a catalase that has at least 75% identity with the amino acid sequence set forth in SEQ ID NO:2, SEQ ID NO:5, or an allelic variant thereof. The invention also relates to a catalase that has at least 85% identity with the amino acid sequence set forth in SEQ ID NO:2, SEQ ID NO:5, or an allelic variant thereof. The invention further relates to a catalase that has at least 95% identity with the amino acid sequence set forth in SEQ ID NO:2, SEQ ID NO:5, or an allelic variant thereof. Catalase activity can be assayed as described herein or by methods known in the art. In addition, the catalase may be lyophilized using methods well known in the art.

In one aspect, the invention relates to a functional fragment of the catalase. In particular, the invention relates to fragments of a catalase, which retain catalase activity and desirable properties, such as, thermal stability, stability at high pH and the absence of inhibition by H₂O₂, as assayed using methods known in the art or disclosed herein. Fragments of a catalase, which retain catalytic activity, include N-terminal truncations, C-terminal truncations, amino acid substitutions, deletions and addition of amino acids (either internally or at either terminus of the protein). For example, conservative amino acid substitutions are known in the art and may be introduced into the catalase of the invention without departing from the scope of the invention.

In another aspect, the invention relates to a catalase or functional fragment thereof derived from an organism. A catalase or functional fragment thereof, is derived from an organism when a nucleic acid or polypeptide from the organism is modified. The nucleic acid or polypeptide may be modified using methods known in the art, such as, mutations or introduction of truncations, substitutions, deletions and/or additions. For example, a nucleic acid derived from Thermus brockianus may be modified by altering the codons of the nucleic acid to reflect codon bias in an appropriate host cell and a catalase derived from Thermus brockianus may be modified by substituting amino acids. However, a sequence derived from an organism retains sufficient homology to the sequence obtained from the organism that an alignment program is capable of identifying the relationship between the starting nucleic acid or polypeptide and the nucleic acid or polypeptide derived from it.

The invention relates to a nucleic acid sequence encoding a thermal tolerant catalase, such as the T. brockianus catalase or an allelic variant thereof. In an exemplary embodiment, the invention relates to the nucleic acid sequence of T. brockianus catalase set forth in SEQ ID NO:1. In one particular embodiment, the invention relates to a nucleic acid that encodes a catalase having at least 85%, 95%, or 98% identity with the amino acid sequence set forth in SEQ ID NO:2 or SEQ ID NO:5. The invention also relates to a nucleic acid that encodes a catalase having at least 95% identity with the amino acid sequence set forth in SEQ ID NO:2 and/or SEQ ID NO:5. The invention further relates to a nucleic acid that encodes a catalase having at least 98% identity with the amino acid sequence set forth in SEQ ID NO:2 and/or SEQ ID NO:5. Catalase activity can be assayed as described herein or by methods known in the art.

An allelic variant denotes any of two or more alternative forms of a gene occupying the same chromosomal locus. Allelic variation arises naturally through mutation, and may result in polymorphism within populations. Gene mutations can be silent (no change in the encoded polypeptide) or may encode polypeptides having altered amino acid sequences. An allelic variant of a polypeptide is a polypeptide encoded by an allelic variant of a gene, wherein the allelic variant of a gene produces a change in the amino acid sequence of the polypeptide encoded therein.

As used herein, “identity” means the degree of sequence relatedness between two polypeptide, or two polynucleotide, sequences as determined by the identity of the match between two strings of such sequences, such as a domain or the complete sequence. Identity may be readily calculated using a number of methods. The term “identity” is well known to those of ordinary skill in the art. Standard methods to determine identity are designed to give the largest match between the two sequences tested. Such methods are codified in computer programs. Preferred computer program methods to determine identity between two sequences include, but are not limited to, GCG (available from Accelrys Inc.), BLASTP, BLASTN and FASTA. The Smith Waterman algorithm may also be used to determine identity.

Polynucleotide sequences having substantial homology or similarity exists when a nucleic acid or fragment thereof will hybridize to another nucleic acid (or a complementary strand thereof) under selective hybridization conditions. Exemplary polynucleotide sequences include those encoding polypeptides having substantial identity to the catalase set forth in SEQ ID NO:2. Selectivity of hybridization exists when hybridization which is substantially more selective than total lack of specificity occurs. Typically, selective hybridization will occur when there is at least about 55% homology over a stretch of at least about nine to 21 nucleotides, preferably at least about 65%, more preferably at least about 75%, and most preferably at least about 93%. The length of homology comparison, as described, may be over longer stretches, and in certain embodiments will include a stretch of at least about 50 nucleotides, more usually at least about 100 nucleotides, typically at least about 200 nucleotides, more typically at least about 400 nucleotides, or at least about 600 or more nucleotides. For example, when comparing a polynucleotide sequence having 860 nucleotides, another polynucleotide sequence having at least 93% identity with the reference sequence is substantially homologous.

Nucleic acid hybridization will be affected by such conditions as salt concentration, temperature, or organic solvents, in addition to the base composition, length of the complementary strands, and the number of nucleotide base mismatches between the hybridizing nucleic acids, as will be readily appreciated by those skilled in the art. Stringent temperature conditions will generally include temperatures in excess of 30° C., typically in excess of 37° C., and most desirably in excess of 45° C. Stringent salt conditions will ordinarily be less than 1000 mM, typically less than 500 mM, and desirably less than 200 mM. However, the combination of parameters is much more important than the measure of any single parameter. The stringency conditions are dependent on the length of the nucleic acid and the base composition of the nucleic acid, and can be determined by techniques well known in the art. See, e.g., Ausubel et al., CURRENT PROTOCOLS IN MOLECULAR BIOLOGY (Wiley, New York, 1994).

Thus, as herein used, the term “stringent conditions” means hybridization will occur if there is at least 75% identity between the sequences. Desirably, there will be at least 85%, more desirably 95%, and most desirably at least 97% identity between the sequences. Such hybridization techniques are well known to those of skill in the art. Stringent hybridization conditions are as defined above and include, but are not limited to, overnight incubation of the probe and target sequences at 42° C. in a solution comprising: 50% formamide, 5×SSC (150 mM NaCl, 15 mM trisodium citrate), 50 mM sodium phosphate (pH7.6), 5× Denhardt's solution, 10% dextran sulfate, and 20 μg/ml denatured, sheared salmon sperm DNA. The filters having the target sequence attached in 0.1×SSC are washed at about 65° C.

If desired, a combination of different oligonucleotide probes may be used for the screening (e.g, screening a recombinant DNA library). The oligonucleotides are labeled (e.g., with ³²P), using methods known in the art, and the detectably-labeled oligonucleotides are used to probe filter replicas from a recombinant DNA library. Recombinant DNA libraries (for example, Thermus cDNA libraries) may be prepared according to methods well known in the art, for example, as described in Ausubel et al., supra. Such libraries may be generated and screened using standard techniques.

In an exemplary embodiment, the invention relates to vectors containing a nucleic acid sequence encoding a catalase of the present invention. The vector may be an expression vector. The invention also relates to a host cell containing a nucleic acid encoding a thermal tolerant catalase. For example, the host cell may be used to express the catalase and may be used as a means of producing the catalase.

Large amounts of the catalase may be produced by recombinant technology, wherein the isolated nucleotide sequence encoding the catalase, or a functional fragment thereof, is inserted into an appropriate vector or expression vector. The vector or expression vector is introduced into an appropriate host cell, which preferably can be grown in large quantities, and the catalase is purified from the host cells or the culture media. The host cells may also be used to supply the catalase of the invention without requiring purification of the catalase (see Yuan, Y.; Wang, S.; Song, Z.; and Gao, R., Immobilization of an L-aminoacylase-producing strain of Aspergillus oryzae into gelatin pellets and its application in the resolution of D,L-methionine, Biotechnol. Appl. Biochem. (2002). 35:107-113). For example, the catalase of the invention may be secreted by host cells, which are contacted with a hydrogen peroxide solution.

Those skilled in the field of molecular biology will understand that any of a wide variety of expression systems may be used to provide the catalase protein. The precise host cell used is not critical to the invention, so long as the host cells produce the catalase when grown under suitable growth conditions. Suitable host cells include, but are not limited to, a eukaryotic host, such as insect cell lines (for example, HIGH FIVE™ from INVIROGEN™ ((BTI-TN-5B1-4), derived from Trichoplusia ni egg cell homogenates), Sf9 or Sf21 cells, Lepidopteran insect cells, mammalian cell lines (for example, primary cell cultures or immortalized cell lines, such as, COS 1, NIH 3T3, HeLa, 293, CHO and U266), transgenic plants, plant cells, Drosophila Schneider2 (S2) cells, Baculovirus Expression Systems, Saccharomyces, Schizosaccharomyces, Pichia, Aspergillus, a prokaryotic host, such as, E. coli, Bacillus, Thielavia terrestris, Acremonium alabamense, Myceliophthora thermophilum, Sporotrichum cellulophilum (see U.S. Pat. No. 5,695,985) or the like. Such cells are available from a wide range of sources (e.g., the American Type Culture Collection, Rockland, Md.; INVITROGEN™; GIBCO™; see also, e.g., Ausubel et al., supra). The method of transformation or transfection and the choice of expression vehicle (vector) will depend on the host system selected. Known transformation and transfection methods are described, e.g., in Ausubel et al., supra; expression vehicles may be chosen from those known in the art (e.g., Cloning Vectors: A Laboratory Manual (P. H. Pouwels et al., 1985, Supp. 1987)).

By way of a general example, the catalase may be cloned into an appropriate vector, such as, pUC based plasmids, Bluescript, or other vectors known in the art. The vector may include regulatory sequences (such as, promoters, enhancers, ribosomal entry sites, transcriptional terminator sequences and polyadenylation sites), additional coding sequences (such as, sequences coding for a fusion protein, a proteolytic cleavage sequences, adaptor sequences or signal sequences) or additional non-coding sequences (such as, introns or restriction sites). The regulatory elements may be native to the catalase of the invention or a heterologous regulatory element. In addition, the vector may include a selectable marker.

The vector may be composed of a single nucleic acid or two or more nucleic acids. Vectors may be linear or closed circular molecules, extrachomosomal or integrated, single copy or multi-copy and may contain one or more origins of replication.

To produce the catalase in a host cell, the nucleic acid sequence encoding a catalase is typically cloned into an expression vector, which may be operably linked to a promoter appropriate for a host cell and may be operatively linked to other transcriptional and translational signals necessary or desirable for expression of the catalase in the host cell. For example, the nucleic acid encoding a catalase of the invention may be placed under the control of a promoter, such as the Saccharomyces inducible metallothionein promoter, a galactose promoter (i.e., Gal 1) or the AOX1 promoter, and introduced into Saccharomyces, or P. pastoris cells or other such cells. Identification of transformed or transfected cells may be accomplished through the use of one or more selectable markers, which are known in the art. In addition, the sequence encoding the catalase may be followed by a poly (A) signal recognized by the host cell.

The catalase of the invention may be expressed as a fusion protein. For example, the catalase gene may be fused in frame to a heterologous amino acid sequence, such as, a histidine or glutathione S-transferase tag, which can then be used to purify the catalase. The heterologous amino acid sequence may include a proteolytic cleavage site. A fusion protein may also include a signal sequence, an ER retention signal, or other sequences known in the art.

In one embodiment, the purified catalase of the invention is substantially free of proteases. Thus, the catalase may be produced in a protease deficient host cell.

An exemplary embodiment of the invention relates to the attachment of the catalase to a water-insoluble solid support (immobilization) (Costa, S. A., Tzanov, T., Paar, A., Gudelj, M., Gubitz, G. M., and Cavaco-Paulo, A., Immobilization of catalases from Bacillus SF on alumina for the treatment of textile bleaching effluents, Enz. Micro. Tech. (2001), 28, 815-819). One aspect of this embodiment allows process water to be passed through or over the immobilized catalase, wherein the catalase converts hydrogen peroxide to oxygen and water without producing undesirable byproducts or contributing the catalase to the process water (Fruhwirth, G. O.; Paar, A.; Gudelj, M.; Cavaco-Paulo, A.; Robra, K.-H.; Gubitz, G. M. An immobilized catalase peroxidase from the alkalothermophilic Bacillus SF for the treatment of textile-bleaching effluents. Appl. Microbiol. Biotechnol. (2002), 60:313-319). Furthermore, immobilization of the enzyme is generally known to increase the stability of the enzyme (see, e.g., illanes, A., Stability Of Biocatalysts, Elect. J. Biotech., (2002), 2(1):1-9). The increased stability may increase the temperatures and pH at which the catalase may be used.

Immobilized catalase can serve as a reusable and removable catalyst and often possess improved storage and operational stability relative to the free catalase. Linking the catalase to a solid support prevents vibration of the catalase and may increase thermal stability. In addition, the microenvironment of the solid support surface may cause a shift in the optimum pH of the catalase. Depending on the charge properties of the support surface, the optimum pH may undergo significant shifts. Id. For example, the optimum pH for the catalase (pH 8.0) when bound to a negatively charged carrier, such as carboxymethylcellulose, may be shifted to higher values, while immobilization on a cationic matrix, such as DEAE-cellulose, may have the opposite effect. Id.

Both chemical and physical methods have been developed for the purpose of immobilizing enzymes. The choice of the solid support and the method of attachment are not critical to the invention and any support or method of attachment known in the art may be used. For example, generally enzymes can be adsorbed onto inert solids, ion-exchange resins, or physically entrapped/encapsulated in solids, such as cross-linked gels, microcapsules, and hollow fibers. The catalase may be covalently bonded to solids via various chemical bonding methods, such as cross-linking, multi-functional reagents, or surface reactive functional groups. Among these methods, chemical covalent bonds traditionally offer the strongest links, and thus the most stable enzyme-solid complexes. To chemically bond enzymes to a solid support, the functional groups on the catalase, through which the covalent bonds are to be formed, and the physical and chemical characteristic of the support material onto which chemically reactive groups are to be attached, should be considered. The functional groups on the amino acids of the catalase that may be utilized for the covalent binding include amino —NH₂ (lysine), carboxylic acid —COOH (aspartic, glutamic), hydroxyl —OH (serine, tyrosine) and cysteine groups. These reactive functional groups, when targeted for covalent bonding attachment to solids, are preferably nonessential for the catalytic activity of the enzymes.

The characteristics of solid supports that are desirable for attachment include, but are not limited to, a large surface area, good chemical, mechanical and thermal stability, hydrophilicity and insolubility. Nonporous materials possess no diffusion constraints, but have very low surface areas for protein binding. The high surface areas of porous materials provide higher protein loading capacity. If most of the surfaces are internal surfaces, however, inefficient diffusion of solutions and the potential for significant pressure changes upstream and downstream of the treatment zone can present major drawbacks. With porous solids, therefore, pore structures may be engineered for efficient diffusion of solutions and, where appropriate, a minimal pressure differential. Natural polymers including polysaccharides (cellulose, cellulose derivatives, dextran, agarose and chitonsan) as well as synthetic polymers, such as polystyrene and polyacrylates, may be used to immobilize the catalase. With most polymers, highly reactive functional groups on the surfaces are typically added to facilitate direct covalent bonding. Reactive natural and synthetic polymers may be prepared with plasma/UV radiation and various chemical and enzymatic reaction mechanisms, such as reductive amination, propoxylation, redox, and transesterification. Thus, the invention utilizes a solid support and a catalase to produce an immobilized catalase, which is useful in the treatment of hydrogen peroxide containing fluids, such as bleach baths in the textile and pulp industries.

The solid support to which the catalase of the invention may be attached may be any molecule or resin that does not prevent catalytic activity under the intended conditions of use. For example, the catalase may be attached via a lysine residue by using a cyanogen bromide-activated Sepharose resin. Further, additional molecules (adaptors) may be added to either the support or the enzyme. In particular, carbon chains or other linkers may be covalently attached between the enzyme and the support molecule.

In an exemplary embodiment, the catalase is immobilized on EUPERGIT® (available from Rohn GmbH, DE), which is a spherical carrier composed of methacrylamide, N,N′-methylene-bis(methylacrylamide) and monomers containing oxirane groups, which can bind enzymes through their amino and sulfhydryl groups. The catalase may be immobilized through amine linkage.

In addition, biosensors based on immobilized catalase have proven to be useful analytical tools for the specific determination of the presence or amount of hydrogen peroxide and the identification of catalase inhibitors, such as cyanides and fluorides. Thus, the invention may be used as an analytical tool or a biosensor.

The catalase of the invention may be purified using chromatography, including, but not limited to ion-exchange, hydrophobic and/or gel filtration chromatography. Under the basic principle of ion-exchange chromatography, the affinity of a substance for the exchanger depends on both the electrical properties of the material and the relative affinity of other charged substances in the solvent. Hence, bound material can be eluted by changing the pH, thus altering the charge of the material, or by adding competing materials, of which salts are but one example. Because different substances have different electrical properties, the conditions for release vary with each bound molecular species. In general, to get good separation, the methods of choice are either continuous ionic strength gradient elution or stepwise elution. A gradient of pH alone is typically not used because it is difficult to set up a pH gradient without simultaneously increasing ionic strength. For an anion exchanger, pH and ionic strength may be gradually increased, or ionic strength alone may be increased. For a cation exchanger, both pH and ionic strength are typically increased. The actual choice of the elution procedure may be determined by a person of skill in the art using known methods. For example, for unstable materials, it is best to maintain fairly constant pH.

Ion exchangers come in a variety of particle sizes, called mesh size. Finer mesh means an increased surface-to-volume ratio and, therefore, increased capacity and decreased time for exchange to occur for a given volume of the exchanger. On the other hand, fine mesh means a slow flow rate, which can increase diffusional spreading. The use of very fine particles, approximately 10 μm in diameter, and high pressure to maintain an adequate flow is called high-performance or high-pressure liquid chromatography or simply HPLC.

High Performance Liquid Chromatography (HPLC) is characterized by a very rapid separation with extraordinary resolution of peaks. Moreover, only a very small volume of the sample is needed because the particles are so small and closely packed that the void volume is a very small fraction of the bed volume. Also, the concentration of the sample need not be very great because the bands are so narrow that there is very little dilution of the sample.

Many substances (e.g., proteins), carry both negative and positive charges and the net charge depends on the pH. In such cases, the primary factor is the stability of the substance at various pH values. Most proteins have a pH range of stability (i.e., where they do not denature) in which they are either positively or negatively charged. For the purpose of discussion herein, the isoelectric point of a protein is the pH at which the protein carries no net charge, below the isoelectric point the protein carries a net positive charge, above it a net negative charge. Hence, if a protein is stable at pH values above the isoelectric point, an anion exchanger is typically used. If a protein is stable at values below the isoelectric point, a cation exchanger is typically used. In addition, other features of the molecule are usually important so that the chromatographic behavior is sensitive to the charge density, charge distribution, and the size of the molecule.

Hydrophobic interaction chromatography (HIC) and reversed-phase chromatography (RPC) are two separation methods based on the interaction between the hydrophobic groups of the sample and an insoluble immobilized hydrophobic molecule, which is typically a short-chain phenyl or octyl non polar group. The mobile phase is usually an aqueous salt solution. In RPC the matrix is typically silica that has been substituted with longer n-alkyl chains, usually C8 (octylsilyl) or C18 (octadecylsilyl).

Separation on HIC matrices are usually done in aqueous salt solutions. Samples are most often loaded onto the matrix in a high-salt buffer and eluted by a descending salt gradient. Alternatively, elution of a protein may be accomplished by increasing the concentration of chaotropic ions in the buffer in a positive gradient, eluting with a positive gradient of a detergent, raising the pH and/or reducing the temperature. Preferably, the catalase is eluted under non-denaturing conditions. HIC depends on surface hydrophobic groups and is carried out under conditions which typically maintain the integrity of the protein (non-denaturing). RPC typically depends on the native hydrophobicity of proteins and is typically carried out under conditions which expose nearly all hydrophobic groups to the matrix (denaturing conditions). However, RPC may be performed under non-denaturing conditions.

Gel filtration chromatography (also known as size-exclusion chromatography or molecular sieve chromatography) may be used to separate proteins according to their apparent size. In gel filtration, a protein solution is passed through a column that is packed with a semipermeable porous resin. The semipermeable resin has a range of pore sizes that determines the size of proteins that can be effectively separated with the column, the fractionation range or exclusion range of the resin.

Proteins larger than the exclusion range of the resin are unable to enter the pores and pass quickly through the column in the spaces between the resin, known as the “void volume” of the column. Small proteins and other low molecular weight substances that are below the exclusion range of the resin enter the pores in the resin and their movement through the column is slowed proportionally to the ability to enter the pores. A protein having a size that falls within the exclusion range of the column will enter only a portion of the pores. The movement of these proteins will be slowed according to their size; smaller proteins will move through the column more slowly because they must pass through a larger volume. Fractions are typically collected as the sample elutes from a column. Larger proteins typically elute in the early fractions and smaller proteins elute in subsequent fractions.

In gel filtration chromatography, proteins are separated roughly according to their molecular weight because this is the major contributor to molecular size. However, the shape of a protein, its quaternary structure and other associated proteins will affect its apparent size in solution. The choice of a chromatography medium is an important consideration in gel filtration. The following is a table showing the exclusion range for some common gel filtration chromatography media.

TABLE 1
Common gel filtration media and exclusion range:

| Gel Filtration Media | Exclusion Range | |
|---|---|---|
| Sephadex G-50 | 1-30 | kD |
| Sephadex G-100 | 4-150 | kD |
| Sephadex G-200 | 5-600 | kD |
| Bio-Gel P-10 | 1.5-20 | kD |
| Bio-Gel P-30 | 2.4-40 | kD |
| Bio-Gel P-100 | 5-100 | kD |
| Bio-Gel P-300 | 60-400 | kD |
| Sephacryl ® 100-HR | 1-100 | kDa |
| Sephacryl ® 200 HR | 5-250 | kDa |
| Sephacryl ® 300 HR | 10-1,500 | kDa |
| Sephacryl ® 400-HR | 20-8,000 | kDa |
| Sephacryl ® 500 HR | 40-20,000 | kDa |

In addition, the catalase may be recovered and purified by methods including, but not limited to, ammonium sulfate or ethanol precipitation, acid extraction, phosphocellulose chromatography, affinity chromatography, hydroxylapatite chromatography, high performance liquid chromatography (HPLC) and lectin chromatography. Protein refolding steps can be used, as necessary, in completing configuration of the mature protein.

The catalase may be purified from a natural source, produced by chemical synthesis, or produced by recombinant techniques from a prokaryotic or eukaryotic host (for example, by bacterial, yeast, higher plant, insect and mammalian cells in culture). In one embodiment, the host cell used to produce a recombinant catalase may post-translationally modify the catalase, for example, by glycosylation or phosphorylation.

The proteins of the invention may be co-translationally, post-translationally or spontaneously modified. For example, by acetylation, farnesylation, glycosylation, myristylation, methylation, prenylation, phosphorylation, palmintolation, sulfation, ubiquitination, and the like. (See, Wold, F. Annu. Rev. Biochem. (1981), 50:783-814).

Two families of catalases are known, one having a heme cofactor, and another structurally distinct family containing non-heme manganese. N-terminal amino acid sequence analysis of the catalase isolated from Thermus brockianus, in combination with other sequence data, produces a sequence alignment with the manganese catalase family, however, unlike typical manganese catalases, the catalase from T. brockianus has surprisingly been found to be inhibited by cyanide.

### EXAMPLE I

### Enzyme Assay

Catalase activity was determined spectrophotometrically by monitoring the decrease in absorbance, at 240 nm, caused by the disappearance of hydrogen peroxide (Beers, R. F., Jr.; Sizer, I. W. A spectrophotometric method for measuring the breakdown of hydrogen peroxide by catalase. J. Biol. Chem. (1952), 195:276-287). The assay was initiated by addition of enzyme solution to 20 mM hydrogen peroxide in 20 mM Tris buffer, at a pH of 8, and was conducted at 70° C., unless otherwise specified. The buffer pH was adjusted to 8 at 70° C. The initial absorbance change (typically the first 30 seconds) was used to calculate the rate of hydrogen peroxide decomposition. The molar absorption coefficient for hydrogen peroxide at 240 nm was assumed to be 43.6 M⁻¹ cm⁻¹ and one unit (U) of catalase activity was defined as the amount of enzyme required to degrade 1 μmol of hydrogen peroxide per minute.

Peroxidase activity of the catalase enzyme was tested using o-dianisidine (0.5 mM) and 2,2′-azino-bis(3-ethylbenzothiazoline-6-sulfonic acid) (10 mM) as substrates with the hydrogen peroxide concentration at 1 mM. The reactions were monitored spectrophotometrically at 460 and 420 nm, respectively. The substrates were dissolved in 20 mM Tris buffer, pH 8.0, and the assays were conducted at 70° C.

### EXAMPLE II

### Isolation and Purification of an Extremely Thermo-alkali-stable Catalase Enzyme from Thermus brockianus

A thermo-alkali-stable catalase enzyme was purified from a thermophilic microorganism, Thermus brockianus. The catalase enzyme was purified via a three step process using ion exchange, hydrophobic interaction, and gel filtration chromatographies. The enzyme was purified to homogeneity as indicated by the presence of a single band on an SDS-PAGE gel.

Microorganism and Culture Conditions: Organisms were obtained from Hot spring LNN2 in Yellowstone National Park, USA, which has an average temperature of 70° C. and an average pH of 7. The GPS coordinates for this site were x=515923.1013974 and y=4931375.3306555 measured on a Tremble GPS Path-finder unit differentially corrected using the Idaho Falls, Id. base station as the reference. Specifics of the GPS unit include Datum=NAD83, PDOP mask=6.0, and minimum satellites=4.

Water, sediment, and fungal mat samples from the spring were collected in sterile 50-mL centrifuge tubes, maintained at approximately 70-80° C. until they could be processed about 4-6 hours after collection. The samples were inoculated into a minimal medium containing 4.2 g/L sodium lactate, 10 mM NH₄Cl, 5.2 mM K₂HPO₄, 0.8 mM MgSO₄.7H₂O, 1.74 mM Na₂SO₄, 25 mg/L MgCl₂, 6.6 mg/L CaCl₂, 2 mg/L MnSO₄, 0.5 mg/L ZnSO₄, 0.5 mg/L boric acid, 5 mg/L FeCl₃, 0.15 mg/L CuSO₄, 0.025 mg/L NaMoO₄, 0.05 mg/L CoNO₃, 0.02 mg/L NiCl₂, 0.08 mg/L pyridoxine hydrochloride, 0.01 mg/L folic acid, 0.1 mg/L thiamine hydrochloride, 0.04 mg/L riboflavin, 0.08 mg/L nicotinamide, 0.08 mg/L p-aminobenzoate, 0.01 mg/L biotin, 0.0004 mg/L cyanocobalamin, 0.08 mg/L D-pan-tothenic acid·Ca, 0.02 mg/L myo-inositol, 0.05 mg/L choline bromide, 0.02 mg/L monosodium orotic acid, and 0.1 mg/L spermidine. Lactate was used as the primary carbon source.

Cultures were grown in 100-mL serum vials at 70° C. on a rotary shaker at 150 rpm. Oxygen levels were tested daily by gas chromatography, and the headspace was flushed with air when oxygen levels fell below 5% (initial oxygen levels started at 21%). Growth was assumed when the cultures became cloudy in appearance, after which cultures were streaked onto agar plates and maintained at 70° C. until growth on the plates occurred. Individual colonies were tested for catalase activity by suspending colonies in a drop of 3% hydrogen peroxide and examining for evolution of bubbles. The isolate showing the highest catalase activity was selected for further characterization.

Microscopic examination of the isolate showed a non-spore-forming, rod-shaped organism. The organism formed diffuse light yellow colonies on agar and was found to be Gram-negative. Sequence analysis (16S rRNA) of this organism identified it as (100% match) Thermus brockianus.

Catalase Purification from Thermus brockianus: Thermus brockianus was cultured to stationary phase at 70° C. using the medium described above in a 100-L B. Braun UE-100D fermentor. The fermentor was run with an impellor speed of 260 rpm, at a pH of 7.2, and at an aeration rate of 30 L/min air that provided between 80% and 100% oxygen saturation (at 70° C.) to the culture. The culture took approximately 100 h to reach stationary phase with a final OD₆₀₀ of 0.38. The cells were collected by centrifugation, resuspended in 20 mM Tris buffer, at a pH of 8, with protease inhibitor (obtained form Sigma Aldrich, St. Louis, Mo.), and disrupted by sonication. The cell debris was removed by centrifugation (34,000×g for 45 min) and the supernatant, containing the crude cell extract, was collected.

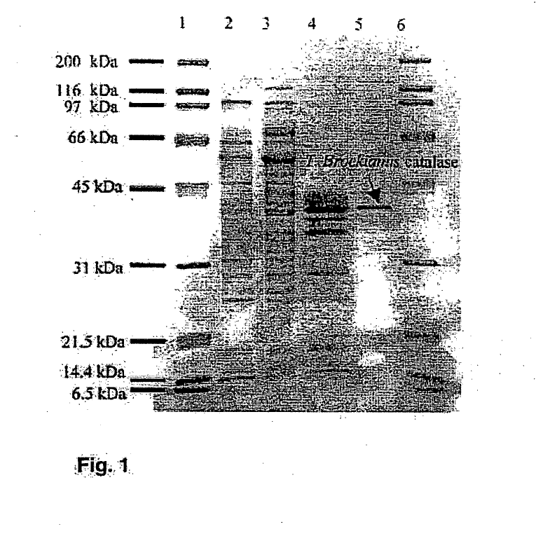

A three-step purification procedure consisting of ion exchange, hydrophobic interaction, and gel filtration chromatography was developed to obtain a highly purified catalase from T. brockianus (FIG. 1).

The crude cell extract was filtered through a 0.2-μm filter and applied to a DEAE ion-exchange column (obtained from Amersham Biosciences, Piscataway, N.J.) equilibrated with 20 mM Tris buffer, pH 8. The enzyme was eluted with a linear gradient from 0 to 500 mM ammonium sulfate in a 100 mM Tris buffer, pH 8 (see, FIG. 1, lane 3). The fractions showing catalase activity were pooled, and the ammonium sulfate concentration of the sample was adjusted to 1.0 M. The sample was then applied to a HiTrap Phenyl Sepharose High Performance hydrophobic interaction column (obtained from Amersham Biosciences, Piscataway, N.J.) equilibrated with 100 mM Tris buffer, at a pH of 8, containing 1 M ammonium sulfate. A decreasing linear elution gradient of ammonium sulfate from 1 M to 0 M was used to elute the enzyme (see, FIG. 1, lane 4). Active catalase fractions were pooled and applied to a Sephacryl S-300 HR gel filtration column (obtained from Amersham Biosciences, Piscataway, N.J.) for the final purification step. The enzyme was eluted from the Sephacryl S-300 HR gel filtration column with 100 mM Tris buffer, at a pH of 8, containing 0.15 M sodium chloride (see, FIG. 1, lane 5). The effectiveness of each purification step was determined by SDS-PAGE using a 12% (w/v) acrylamide gel (FIG. 1). Protein concentrations were determined using the DC protein assay (obtained from Bio-Rad; Hercules, Calif.) with bovine serum albumin as a standard.

The effectiveness of each purification step is given in Table 2 and FIG. 1. The three-step procedure described here resulted in 1160 total units of catalase activity, 65-fold purification of the crude cell extract, and a specific catalase activity of 5300 U/mg of protein, with a yield of 0.8%. The 65-fold purification achieved in this procedure is comparable to that obtained with other bacterial catalases. While the yield from this purification method was less than other published protocols, this method had the advantages of being rapid and yielding a very pure catalase enzyme as evidenced by the presence of a single band after the gel filtration step (FIG. 1, Lane 5).

Alternative methods may be used to purify the catalase and further optimization of the protocol used herein may be made by a person of skill in the art.

TABLE 2
Purification of the catalase from T. brockianus:

| | Total | Total | | | |
|---|---|---|---|---|---|
| | Activity | Protein | Specific | Yield | Purification |
| | (U) | (mg) | Activity | (%) | (fold) |
| Crude Cell | 139,200 | 1,700 | 82 | 100 | 1.0 |
| Extract | | | | | |
| Ion Exchange | 25,440 | 153 | 166 | 18 | 2.0 |
| Hydrophobic | 1,440 | 2.4 | 600 | 1.0 | 7.3 |
| Interaction | | | | | |
| Gel Filtration | 1,160 | 0.22 | 5,320 | 0.8 | 65 |

### EXAMPLE III

### Characterization of the Thermo-alkali-stable Catalase Enzyme from Thermus brockianus

Molecular Mass: The molecular mass of the purified catalase was estimated via gel filtration under native nondenaturing conditions using molecular mass standards (obtained from Amersham Biosciences; Piscataway, N.J.). The sizing column was run under the same conditions as used with the Sephacryl S-300 HR gel filtration column for purification. The subunit size of the catalase was estimated from SDS-PAGE gel electrophoresis on a 12% acrylamide gel using molecular mass standards obtained from Bio-Rad Laboratories, Hercules, Calif. (FIG. 1, lane 5). Proteins were visualized on the gel using SimplyBlue SafeStain (obtained from Invitrogen Corp.; Carlsbad, Calif.).

SDS-PAGE of the purified catalase enzyme showed a single band corresponding to a subunit size of 42.5 kDa. The gel filtration results showed an approximate native protein molecular mass of 178 kDa. Indicating that the enzyme is composed of four identical subunits. The subunit and native enzyme sizes for this enzyme are significantly smaller than those reported for other tetrameric catalase enzymes (i.e., Bacillus sp. with a subunit size of 70.5 kDa and catalase size of 282 kDa; E. coli with a subunit size of 84.3 kDa and a catalase size of 337 kDa; Rhodobacter capsulatus with a subunit size of 59 kDa and a catalase size of 236 kDa; and Neurospora crassa with a subunit size of 80 kDa and a catalase size of 320 kDa).

Kinetics: The Michaelis-Menten constants for the enzyme were determined using the standard assay with hydrogen peroxide concentrations ranging from 3 to 450 mM. The constants were calculated by fitting the Michaelis-Menten equation to a plot of reaction velocity versus substrate concentration using nonlinear analysis (using GraFit Version 4, Erithacus Software Limited, Horley Surrey, U.K.). Irreversible inhibition of the catalase enzyme was tested using 40 mM 3-amino-1,2,4-triazole and 1 mM cyanide. The enzyme was assayed as in Example I, except that it was preincubated with the inhibitor for 5 min prior to assay.

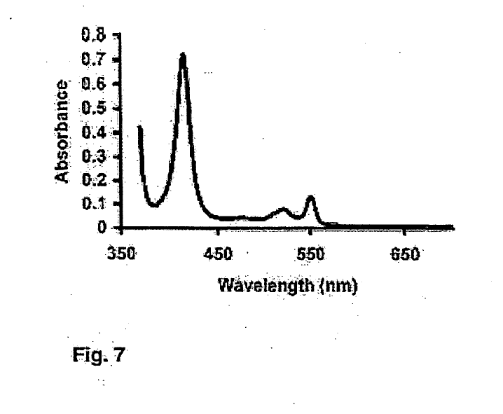

The rate of hydrogen peroxide decomposition as a function of hydrogen peroxide concentration is shown in FIG. 5 for the T. brockianus catalase. Nonlinear curve fitting of the data to the Michaelis-Menten equation yielded a Kₘ of 35.5 mM and a Vₘₐₓ of 20.3 mM/min·mg protein, which corresponds to a turnover number (k_{cat}) of 3.6×10⁵ min⁻¹ and a catalytic efficiency (k_{cat}/Kₘ) ratio of 1.7×10⁵ s⁻¹ M⁻¹. The turnover number was calculated assuming four active centers per catalase molecule. A comparison of kinetic parameters of catalase enzymes from various sources is given in Table 3. The Kₘ value for the T. brockianus catalase is lower than that reported for the thermostable catalase from T. aurantiacus but higher than the Kₘ values reported for most other catalases.

TABLE 3
Comparison of kinetic parameters for catalase and catalase-
peroxidases from various organisms:

| | | Vₘₐₓ | | | |
|---|---|---|---|---|---|
| | | (mM | | | H₂O₂ |
| | Kₘ | min⁻¹ | | K_{cat}/Kₘ | Inhibition |
| Source | (mM) | mg⁻¹) | K_{cat} (min⁻¹) | (M⁻¹ s⁻¹) | (mM) |
| T. aurantiacus | 48.0 | NR^{a} | 6.4 × 10⁶ | 2.2 × 10⁶ | 60 |
| T. album | 15.0 | 2.3 | NR | NR | 20 |
| Bacillus sp. | 6.8 | NR | NR | NR | 30 |
| E. coli | 3.9 | NR | 9.8 × 10⁵ | NR | NR |
| R. capsulatus | 4.2 | NR | NR | NR | NR |
| N. crassa | 25.0 | NR | NR | 4.57 × 10⁶ | None^{b} |
| Vitreoscilla sp. | 16.0 | NR | 1.6 × 10⁶ | 2.70 × 10⁷ | NR |
| M. tuberculosis | 5.2 | NR | 6.06 × 10⁵ | 1.95 × 10⁶ | NR |
| A. nidulans | 4.3 | NR | 4.3 × 10⁵ | 1.66 × 10⁶ | 10 |
| T. brockianus | 35.5 | 20.3 | 3.6 × 10⁵ | 1.7 × 10⁵ | None^{c} |

^{a}NR: Not Reported
^{b}No inhibition was observed for H₂O₂ concentrations up to 200 mM.
^{c}No inhibition was observed for H₂O₂ concentrations up to 450 mM.

Isoelectric Point: The isoelectric point (pI) of the enzyme was determined using a model 111 Mini Isoelectric Focusing Cell from Bio-Rad Laboratories (Hercules, Calif.). A 5% (w/v) acrylamide gel was focused for 15 min at 100 V, 15 min at 200 V, and 60 min at 450 V. After focusing was complete, the gel was removed from the cell and cut in half. Proteins were visualized on one-half of the gel by staining with SimplyBlue SafeStain (obtained from Invitrogen Corp.; Carlsbad, Calif.). On the other half of the gel, hydrogen peroxide solution was added to locate the catalase activity indicated by the evolution of bubbles. The single band of catalase identified was compared to pI standards ranging from 4.45 to 9.6 (obtained from Bio-Rad; Hercules, Calif.).

The isoelectric point of the catalase was determined to be 4.7. The measured isoelectric point for the T. brockianus catalase was comparable to those reported for catalases and catalase-peroxidases from Halobacterium halobium of 4.0, Thermoascus aurantiacus of 4.5, Vitreoscilla sp. of 5.0 and 5.2, and Anacystis nidulans of 4.7.

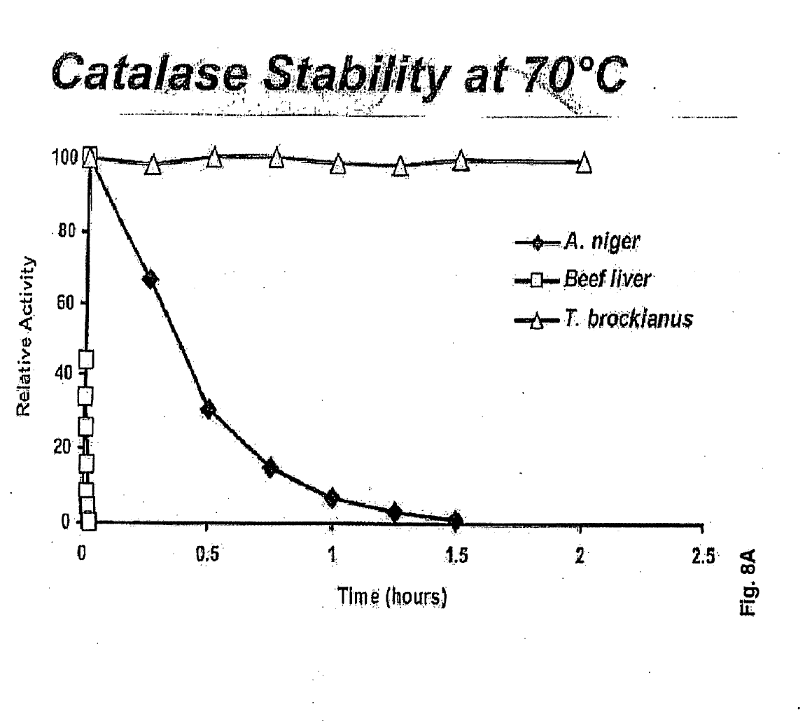

Spectral Characteristics: The absorption spectra of the native enzyme, enzyme reduced with 1 mM sodium dithionite, and enzyme treated with 10 mM KCN were measured (FIG. 6). The enzyme preparation used in the spectral analysis showed a single band by SDS-PAGE analysis, however, as will be recognized by a person of ordinary skill in the art, there still exists the possibility of a contaminating protein. The protoheme type and content were determined through the formation of a pyridine hemochrome as described by Falk (Falk, J. E. Porphyrins and Metalloporphyrins; Elsevier: Amsterdam, 1964). All spectra were measured at both 22° C. and 70° C. to examine possible conformational changes by the enzyme at those temperatures. The molar absorption coefficient for the pyridine hemochrome was assumed to be 191.5 mM⁻cm⁻¹ (Id.).

### EXAMPLE IV

### Classification

The T. brockianus catalase was classified as a monofunctional catalase based on inhibition studies. In particular, the T. brockianus catalase was completely inhibited by 40 mM 3-amino-1,2,4-triazole. Since this compound is a classic inhibitor of monofunctional catalases, while catalase-peroxidases are insensitive to it, this serves to classify the T. brockianus catalase as monofunctional. The classification was confirmed by the absence of peroxidase activity using the peroxidase substrates o-dianisidine and 2,2′-azino-bis(3-ethylbenzothiazoline-6-sulfonic acid). Another common catalase inhibitor is potassium cyanide, which showed 91% inhibition of T. brockianus catalase activity at a concentration of 1 mM. However, potassium cyanide also inhibits catalase-peroxidases and, therefore, does not distinguish the two classes.

### EXAMPLE V

### Optimum Temperature, Optimum pH, and Stability

To determine pH response, suitable buffers covering the pH range from 4 to 11 were used, which include: 50 mM sodium citrate (pH 4-6), 50 mM potassium phosphate (pH 7), 50 mM Tris (pH 8-9), and 50 mM glycine (pH 10-11). The assays were conducted at 70° C. with 20 mM hydrogen peroxide in the appropriate buffer, and the pH of the buffers was adjusted to the correct value at that temperature. The enzyme assay was conducted as described herein.

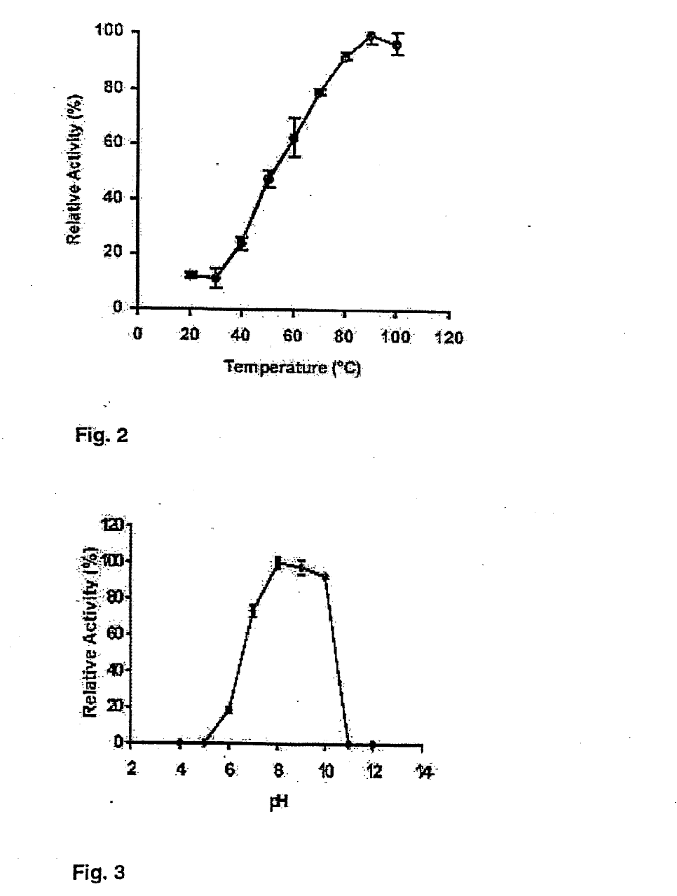

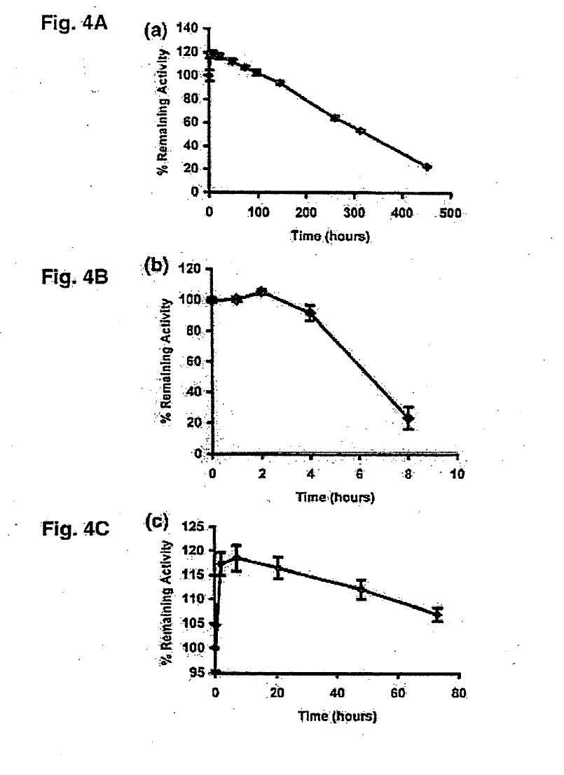

The optimum temperature for enzyme activity was determined by assaying the enzyme activity using the protocol described in Example I at temperatures ranging from 20 to 94° C. (FIG. 2). For example, temperature stability of the catalase enzyme was examined by incubating a 0.1 mg/mL enzyme solution at 80 or 90° C. and periodically removing samples. A mineral oil overlay was placed on top of the enzyme solution to prevent evaporation. Enzyme stability as a function of pH was assessed using a 1 mg/mL solution of catalase enzyme in buffers that are appropriate to maintain a pH of 9, 10, or 11. The catalase enzyme was incubated at an appropriate temperature, for example 70° C., and samples were periodically removed and tested (FIG. 3).

The activity of the catalase, as a function of temperature and pH, is shown in FIGS. 2 and 3. The enzyme had limited activity at 20° C., with activity increasing as the temperature increased, up to a maximum activity at 90° C. The T. brockianus catalase also had activity over a broad pH range of 6-10, with the maximum activity at pH 8. Stability of the T. brockianus catalase was also measured at alkaline pHs ranging from 9 to 11 at 70° C.

The stability of the T. brockianus catalase was determined under numerous conditions, including, 25° C. and a pH of 10 and 11; 70° C. and a pH of 9, 10 and 11; 75° C. and pH 8; 90° C. and pH 8. The enzyme was extremely stable under all conditions, with 70° C. and pH 11 having the shortest half-life. The enzyme had half-lives of 510 and 360 h (21 and 15 days) at 70° C. and a pH of 9 and 10, respectively. At a pH of 11 and 70° C., the stability of the T. brockianus catalase was reduced, with complete loss of activity after 30 min. The half-lives for the enzyme at 25° C. were 44 days at pH 10 and 100 days at pH 11. The half-life for the enzyme at 80C and a pH of 8 was 13.8 days, and the half-life at 90° C. and a pH of 8 was 3 hours.

Thus, the invention is particularly useful in the conversion of hydrogen peroxide to oxygen and water in any situation where the reaction may be conducted at a high temperature or high pH. In particular, the stability of the catalase of the invention, as a function of pH and temperature was determined to be higher than the stability of other catalases under similar conditions.

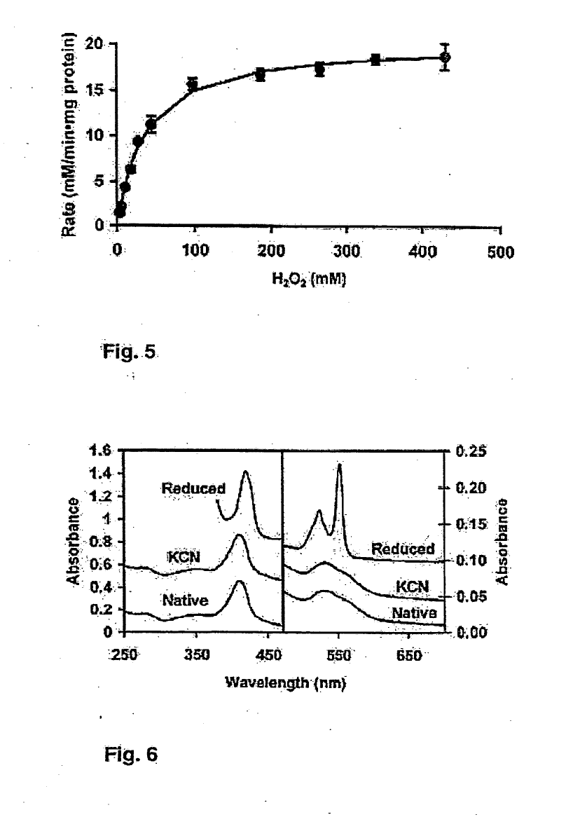

Effect on the Activation of T. brockianus catalase by storage at 4° C.: Stability of the T. brockianus catalase was tested at both 80 and 90° C. (FIGS. 4a and b) at the optimum pH (8) for activity. An unexpected activation effect of the catalase from T. brockianus was observed during the stability studies (FIG. 4c). At 80° C., the activity increased approximately 20% over the initial activity in the first 7 h of incubation, and at 90° C., a 5% increase in activity was observed over the first 2 h of incubation (FIG. 4b). The increase in activity is believed to result from storing the enzyme at 4° C. prior to the assay used to obtain the initial activity. While at 4° C., the enzyme is believed to have been configured into a less active state that was maintained during the initial assay. Although the enzyme was heated to 70° C. for 3 min prior to addition to the assay, this did not appear to be enough time to reactivate the enzyme to the more active state. When the enzyme was incubated at elevated temperatures, the enzyme configuration is believed to gradually change to a more active state, such that subsequent assays of activity showed higher initial activity levels. This reactivation was temperature-dependent, since the activity took longer to peak at 80° C. (7 h, FIG. 4c) compared to 90° C. (2 h, FIG. 4b).

A similar activation of mesophilic catalase enzymes from Rhodospirillum rubrum and Micrococcus luteus has been reported, with activations of 88% and 55% above the initial activity, respectively, after 5 min of incubation at 50° C. The authors attributed this effect to a reversible conformation change in the enzyme. The effect on the mesophilic catalase was also determined to be temperature-dependent, with the amount of activation increasing with increasing temperature up to 50° C. and then decreasing with further increases in temperature. The activation effect was much more rapid in the mesophilic catalases, with activation being observed after 5 min of incubation and starting to decline after 15 min of incubation, compared to the 2-7 h required for the activation effect to peak in the T. brockianus catalase. This may be due to the physical nature of thermostable enzymes since they tend to be more rigid than their mesophilic counterparts and may take longer to reconfigure to the higher activity level. The inventors observed that the activation effect did not occur in catalase-peroxidase enzymes from E. coli and Rhodopseudomonas capsulata. Because of the activation effect observed, the T. brockianus catalase half-lives at 80 and 90° C. were calculated using the data obtained after the full activation had occurred.

The T. brockianus catalase was also extremely stable when stored in 20 mM Tris, pH 8 at 4° C., with no apparent loss of activity after 2 years of storage.

Inhibition by hydrogen peroxide: Many catalases do not show true Michaelis-Menten behavior (i.e., saturation at high substrate levels) because of inactivation/inhibition of the enzyme by hydrogen peroxide at fairly low concentrations (Table 3). In contrast, the T. brockianus catalase demonstrated saturation kinetics at hydrogen peroxide concentrations above 50 mM. There was no apparent substrate inhibition/inactivation of the catalase enzyme at hydrogen peroxide concentrations up to 450 mM, the limit of the spectrophotometric assay. However, this inhibition/inactivation assay was conducted over a relatively short time frame, thus long term effects on inactivation were not deteremined. In contrast, the catalases from T. aurantiacus and T. album both show substantial substrate inhibition at 60 and 20 mM hydrogen peroxide, respectively. Thus, the catalase of the invention functions in the presence of high concentrations of substrate.

### EXAMPLE VI

### Spectral Characterization of T. brockianus Catalase

The absorption spectra of the T. brockianus catalase preparation, treated with 1 mM sodium dithionite, and catalase treated with 10 mM KCN is shown in FIG. 6. The catalase had virtually no absorbance at 280 nm, suggesting that the enzyme has few aromatic amino acids. The catalase showed a strong Soret peak at 410 nm and a peak at 534 nm with a shoulder occurring from 560 to 570 nm. While applicants do not wish to be bound by any theory, this data may suggest that the T. brockianus catalase is a heme catalase, rather than a Mn-catalase, since the absorbance spectra of Mn-catalases completely lack the Soret peak. In addition, the Soret peak of the T. brockianus is red shifted compared to the more typical 406 nm Soret peak for other catalases, although a Soret peak at 408 nm has been reported. Further, the spectral data for the T. brockianus catalase preparation lacks the typical heme charge-transfer bands at 505 and 624 nm that are distinctive of high spin ferric heme proteins and instead has a broad peak centered at 534 nm with a shoulder from 560 to 570 nm that is more typical of heme protein spectra in a low spin configuration.

Since the T. brockianus catalase preparation in the native resting state has a spectrum typical of a low spin state, the spectral analysis is consistent with the distal 6-coordinate position of this enzyme being filled with a ligand that results in the low spin state. Results obtained from site-directed mutagenesis of the proximal His/Trp/Asp of a catalase-peroxidase from the cyanobacteria Synechocystis supports this assertion. Mutants with a 6-coordinate low spin heme state were indicated by a slight red shifting of the Soret peak from 406 nm to 410-416 nm, a peak at about 530 nm, and either an absent or weak peak at 630 nm. These alterations of the absorbance spectra are very similar to the spectrum obtained for the T. brockianus catalase. A 6-coordinate heme iron may also explain the relatively lower activity of the T. brockianus catalase compared to that of other catalases, since the 6-coordinate mutants are much less active than the wild-type enzyme. The spectrum obtained in the presence of 10 mM KCN is also consistent with the 6-coordinate heme hypothesis since the spectrum obtained was identical to the native enzyme preparation with no shift in the Soret peak and no changes in the minor peak at 534 nm or the 560-570 shoulder.

Catalases with a vacant distal heme position exhibit a Soret peak shift of approximately 15-20 nm when cyanide binds in that position. Since this shift was not observed in the catalase preparation, this data is consistent with cyanide being blocked from binding at this site, as would occur if the site were already occupied. Similarly, α and β bands at 555 nm and 580-590 nm that are seen when cyanide binds to the distal heme position were not observed in the T. brockianus catalase preparation's cyanide spectrum. This result was unexpected because this catalase was strongly inhibited by cyanide. It is widely accepted that cyanide acts to inhibit catalases through binding in the distal heme position, which blocks the active site of the enzyme. The spectral analysis is consistent with the cyanide not binding in this location of the T. brockianus catalase. Thus, the spectral data suggests that cyanide inhibition of the enzyme may occur through some other mechanism and the T. brockianus catalase has an active site different than the typical catalase. There are heme proteins that do possess 6-coordinate heme iron, an example is cytochrome c peroxidase that has a heme c with thiolate and imidazole groups in the 5- and 6-coordinate positions; however, there have been no previous reports of a naturally occurring 6-coordinate catalase enzyme. An alternative explanation of the spectral data is that the enzyme was in an inactive state during measurement of the spectra. The above spectra were taken at 22° C., a temperature where the enzyme has virtually no activity. The activation phenomena described above also supports the assertion that the enzyme is locked into a nonactive state at lower temperatures. It is possible that the nonactive state of the enzyme is the 6-coordinate heme configuration observed from the spectra. Alternatively, the presence of a contaminating heme c containing protein may produce the spectral properties obtained, however, SDS-PAGE analysis of the enzyme preparation used throughout the spectral analysis only showed a single band.

To demonstrate that the active enzyme was not in the inactive state during measurement of the absorbance spectra, the native and KCN spectra were measured again at 70° C. after a 2-h incubation at 80° C. These spectra were identical to those obtained at the lower temperature, consistent with the idea that T. brockianus catalase is also in a 6-coordinate low spin state while in the active configuration. The T. brockianus catalase preparation was reduced with sodium dithionite, resulting in a shift of the Soret peak to 419 nm, loss of the 534 nm peak, and appearance of peaks at 523 and 553 nm. This behavior was also surprising because monofunctional catalases are generally very resistant to reduction, whereas catalase-peroxidases are easily reduced with dithionite. Although most of the properties of the T. brockianus catalase are consistent with monofunctional catalases, the spectral analysis of the catalase preparation is consistent with the catalase having at least one property that has only been seen previously in catalase-peroxidase enzymes. The same spectrum was also acquired at 70° C. to ensure that the observed effect were not an artifact of the original scan conditions. The same results were obtained at both temperatures.

Although no previously reported monofunctional catalases have been shown to have properties of both types of enzymes, there has been one report of a recombinant catalase-peroxidase cloned from the putative perA gene of Archaeoglobus fulgidus that also had a property previously only seen in monofunctional catalases. This recombinant enzyme demonstrated the classic behavior of catalase-peroxidases with both catalatic and peroxidative activity, a sharp pH optimum for activity, rapid inactivation in the presence of hydrogen peroxide, and was easily reducible by dithionite. However, the enzyme was inhibited by 3-amino-1,2,4-triazole, a property previously attributed only to monofunctional catalases.

Treatment of the T. brockianus catalase with pyridine/NaOH and sodium dithionite produced a spectral pattern of a pyridine hemo-chrome with spectral peaks at 415, 521, and 550 nm (FIG. 6). Most reported catalases utilize protoheme IX as the heme group in the enzyme, which have pyridine hemochrome absorption peaks at 418, 526, and 556 nm. The peaks observed in the T. brockianus pyridine hemochrome spectrum are slightly shifted from those peaks. If it is assumed that the T. brockianus catalase possesses a protoheme IX and the protoheme content is calculated from the absorption of the pyridine hemochrome peak at 415 nm, a value of 6.7 molecules of protoheme IX per molecule of catalase is obtained. This level of heme would be the highest reported for any catalase enzyme, where more typical levels are 2-4 molecules of heme per molecule of catalase. This high level is consistent with the uncharacteristically high Reinheitzal number (A410/A275) of 2.8 compared to more typical ratios of 0.5-1.0.

However, the fact that the T. brockianus pyridine hemochrome peaks are shifted from typical protoheme IX peaks is consistent with the T. brockianus catalase utilizing another type of heme group in its active site. The T. brockianus spectrum closely resembles the pyridine hemochrome spectra of heme c. The presence of heme c in the T. brockianus catalase is also consistent with the 6-coordinate heme of the catalase, since it has been reported that cytochrome c peroxidase containing a heme c group is 6-coordinate. Using the molar absorption coefficient for heme c, 29.1 mM⁻¹ cm⁻¹ (Falk, 1964) for the absorption peak at 550 nm, there are calculated to be approximately eight molecules of heme c per molecule of catalase (two per subunit).

### EXAMPLE VII

### Immobilization of Catalase

Primary amine groups of a solid support media, for example, controlled pore glass (CPG) CPG-NH2, are activated with glutaraldehyde by incubation of the granules in about 2.5% glutaraldehyde for an appropriate period of time, for example, 1 hour, at room temperature. The support media is washed with phosphate buffer and incubated with a BSA solution. The excess BSA is removed by washing with buffer and the support is incubated with a catalase containing solution.

In another exemplary embodiment, the catalase was immobilized onto Eupergit® C beads as follows: Enzyme solution made up into 50 mM phosphate buffer, pH 7.2 was prepared at 12,500 Units of activity. Four mL of this solution was added to 1 g of Eupergit® C beads and allowed to incubate at room temperature for 48 hours. The beads were washed to remove any unreacted enzyme with 40 mL of 50 mM phosphate buffer, pH 7.2. Four mL of 1M glycine buffer, pH 7.4, was added to the beads and allowed to incubate at room temperature for 24 hours. This step served to block any unreacted sites on the Eupergit® C beads. The beads were then washed with 100 mL of 50 mM phosphate buffer, pH 7.2 and another 100 mL of double distilled water. Finally, the beads were resuspended in 100 mM phosphate buffered saline and stored at 4° C. All wash solutions were assayed for enzyme activity to determine the amount of enzyme bound to the beads.

Enzyme Kinetics. Kinetic parameters for the enzyme in solution were determined using the assay described herein and hydrogen peroxide concentrations ranging from 1.5 to 500 mM. Assays were run at the optimum temperature and pH for activity of each enzyme. The Michaelis Menten parameters, Km and Vmax, were determined by fitting the velocity versus substrate concentration curve to the Michaelis-Menten equation using non-linear analysis (GraFit; Erithacus Software Limited, Horley Surrey, UK). Kinetic parameters for immobilized enzyme were determined using the method of Lilly et al., 1966. Immobilized enzyme was packed into a 4.6 mm I.D. by 10 cm length column. The column was equilibrated with 100 mM phosphate buffered saline (PBS), pH 7.2 and 0.15 M NaCl, at 20 mL/min. Various concentrations of hydrogen peroxide were then introduced to the column at 20 mL/min. After equilibrium was reached, effluent from the column was sampled and the absorbance at 240 nm was measured to determine the hydrogen peroxide concentration. Hydrogen peroxide concentrations used ranged from 10 mM to 200 mM. The columns were maintained at the optimum temperature for activity of each enzyme during the runs.

The apparent Michaelis-Menton constant, Km, for three enzymes were determined for both immobilized and non-immobilized enzyme. For all three enzymes, the Kₘ values were less for the immobilized enzyme than for the enzymes in solution (see, Table 4). It is not clear why this is the case since diffusional resistances introduced by the solid phase generally results in increased apparent Km values (Bailey and Ollis, 1986).

Kₘ values for immobilized catalase:

| | Approximate Michaelis-Menten Constant Kₘ (mM) | |
|---|---|---|
| Catalase Source | Non-Immobilized Enzyme | Immobilized Enzyme |
| Aspergillus niger | 439^{a} | 169 |
| Beef Liver | 37^{b} | 22 |
| Thermus brockianus | 35 | 17 |

^{a}Approximated value - saturation kinetics were not observed at substrate concentrations up to 500 mM; and
^{b}Approximated value - inhibition observed at substrate concentrations above 100 mM.

Results from the immobilized enzyme studies showed increases in enzyme stability when immobilized. For beef liver catalase, the stability at 70° C. and pH 10 increased from no stability to being stable for 5 minutes. The A. niger catalase was stable for at least 6 hours under these conditions. The stability of immobilized T. brockianus catalase is assayed as done with immobilized beef liver catalase. Long term stability of immobilized T. brockianus catalase is believed to be increased as well.

### EXAMPLE VIII

### The Immobilized Catalase is Used to Remove H₂O₂ from Process Water

Bleaching of pulp may be conducted at a temperature between about 40 and about 70° C., but may be conducted up to 100° C. Furthermore, the pH-value of the stabilized aqueous hydrogen peroxide solutions may range from about 9 to about 13.

In an exemplary embodiment, the process water from the bleaching process is shunted from the bleaching chamber and conveyed to a rechargeable column having the immobilized catalase. The process water is controllably passed through the immobilized catalase column at an appropriate rate, which may be dependent on pH, temperature and hydrogen peroxide concentration. The effluent from the immobilized catalase column may be treated to remove any other components and/or tested to determine the remaining concentration of hydrogen peroxide. The effluent may be reused in the bleaching process or may be used in a subsequent dyeing process.

In another exemplary embodiment, where the pH of the process water is between about 11 and about 13, the pH may be adjusted by the addition of an acid, such as a phosphonic acid, prior to passing the process stream over the immobilized catalase column.

### EXAMPLE IX

### The Immobilized Catalase is Used to Remove H₂O₂ from Process Water

The catalase is attached to a resin having an appropriate density. The immobilized catalase and process stream are added to a container, which may be stirred during conversion of the hydrogen peroxide to maintain contact between the process stream and the immobilized catalase.

The container or upstream components may contain heating or cooling elements to establish or maintain a desirable temperature. For example, a process stream at a temperature above 90° C. may be cooled to a temperature between 60° C. and 90° C. before, or as, it is added to the container. In one exemplary embodiment, the temperature of the process stream is between about 80° C. and about 90° C.

Likewise, the pH of the process stream may be adjusted. In particular, the pH may be adjusted to a pH of between about 7.5 and about 11. In one embodiment, the pH is adjusted to between about 8 and about 9.

Following conversion of the hydrogen peroxide, the immobilized catalase is allowed to settle out of the processing stream. The water of the processing stream is withdrawn from the container. Additional process stream may be added to the immobilized catalase and the process repeated.

### EXAMPLE X

N-terminal sequencing of the catalase of the invention was conducted. The N-terminal amino acids of the first sequencing were identified as MFLRIDRLQIELPM(P)KEQDP NAA (SEQ ID NO:3), and the N-terminal amino acids of a second amino acid sequencing reaction were identified as MFLRIDRLQIELPPPPE (SEQ ID NO:4).

Sequencing of the T. brockianus genome identified the polypeptide of SEQ ID NO:2. Comparison of the three amino acid sequences from the genomic sequencing and N-terminal amino acid sequencing of the catalase indicates that the genomic nucleic acid sequence corresponds to the isolated catalase. Therefore, the N-terminus of SEQ ID NO:5 includes the first five amino acids as identified in the N-terminal amino acid sequencing runs (see, alignment below).

| | |
|---|---|
| D R L Q I E L P M P K E Q D P N A A A A | Genomic nucleic acid sequence |
| M F L R I D R L Q I E L P M P K E Q D P N A A | 1^{st} N-terminal AA sequencing |
| M F L R I D R L Q I E L P P P P E | 2^{nd} N-terminal AA sequencing |
| - - - - - - - - - - - - - X - X - - - - - - - | Consensus |

The sequence homology of SEQ ID NO:2 and/or SEQ ID NO:5 to the manganese catalase family is surprising in view of the inhibition by cyanide.

The catalase of the invention (e.g., the catalase from T. brockianus) has exceptional stability at elevated temperatures and pH compared to that of many other reported catalase enzymes. The high temperature and pH stability of the T. brockianus catalase makes the enzyme useful, for example, in the treatment of industrially generated hydrogen peroxide process streams. In addition, this catalase has a number of unusual features compared to those of other reported catalases. The enzyme shares most of the features common to monofunctional catalases such as a broad pH optimum, no peroxidative activity, and inhibition by 3-amino-1,2,4-triazole; yet the enzyme was easily reduced by dithionite, a property previously only observed in catalase-peroxidase enzymes. Other unusual properties observed in the T. brockianus catalase included the spectral data, inhibition by cyanide and sequence conservation with manganese catalases, which are not typically inhibited by cyanide.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### REFERENCES

All references, including publications, patents, and patent applications, cited herein are hereby incorporated by reference to the same extent as if each reference were individually and specifically indicated to be incorporated by reference and were set forth in its entirety herein, including the following references:
Allgood, G. S.; Perry, J. J., Characterization of a manganese-containing catalase from the obligate thermophile Thermoleophilum album. J. Bacteriol. (1986), 168(2):563-567;
Apitz, A.; van Pee, K. H., Isolation and characterization of a thermostable intracellular enzyme with peroxidase activity from Bacillus sphaericus. Arch. Microbiol. (2001), 175:405-412;
Beers, R. F., Jr.; Sizer, I. W., A spectrophotometric method for measuring the breakdown of hydrogen peroxide by catalase. J. Biol. Chem. (1952), 195:276-287;
Cloning Vectors: A Laboratory Manual (P. H. Pouwels et al. edt., 1985, Supp. 1987;
Costa, S. A., Tzanov, T., Paar, A., Gudelj, M., Gubitz, G. M., and Cavaco-Paulo, A., Immobilization of catalases from Bacillus SF on alumina for the treatment of textile bleaching effluents, Enz. Micro. Tech. (2001), 28, 815-819;
Falk, J. E., Porphyrins and Metalloporphyrins; Elsevier: Amsterdam, 1964;
Fruhwirth, G. O.; Paar, A.; Gudelj, M.; Cavaco-Paulo, A.; Robra, K.-H.; Gubitz, G. M. An immobilized catalase peroxidase from the alkalothermophilic Bacillus SF for the treatment of textile-bleaching effluents. Appl. Microbiol. Biotechnol. (2002), 60:313-319;
Gudelj, M.; Fruhwirth, G. O.; Paar, A.; Lottspeich, F.; Robra, K. H.; Cavaco-Paulo, A.; Gubitz, G. M., A catalase-peroxidase from a newly isolated thermoalkaliphilic Bacillus sp. with potential for the treatment of textile bleaching effluents. Extremophiles (2001), 5:423-429;
Illanes, A., Stability Of Biocatalysts, Elect. J. Biotech., (2002), 2(1): 1-9;
Kagawa, M.; Murakoshi, N.; Nishikawa, Y.; Matsumoto, G.; Kurata, Y.; Mizobata, T.; Kawata, Y.; Nagai, J., Purification and cloning of a thermostable manganese catalase from a thermophilic bacterium. Arch. Biochem. Biophys. (1999), 363(2):346-355;
Kengen, S. W. M.; Bikker, F. J.; Hagen, W. R.; de Vos, W. M.; van der Oost, J. Characterization of a catalase-peroxidase from the hyperthermophilic archaeon Archaeoglobus fulgidus. Extremophiles (2001), 5:323-332;
Lilly, M. D., Hornby, W. E., and Crook, E. M. The kinetics of carboxymethylcellulose-ficin in packed beds. Biochem. J. (1966) 100:718.
Loprasert, S.; Negoro, S.; Okada, H. Thermostable peroxidase from Bacillus stearothermophilus. J. Gen. Microbiol. (1988), 134:1971-1976;
Paar, A.; Costa, S.; Tzanov, T.; Gudelj, M.; Robra, K.-H.; Cavaco-Paulo, A.; Gubitz, G. M. Thermo-alkali-stable catalases from newly isolated Bacillus sp. for the treatment and recycling of textile bleaching effluents. J. Biotechnol. (2001), 89:147-153;
Scientific Committee On Toxicity, Ecotoxicity And The Environment (CSTEE) (EINECS No.:231-765-0);
Wang, H.; Tokusige, Y.; Shinoyama, H.; Fujii, T.; Urakami, T. Purification and characterization of a thermostable catalase from culture broth of Thermoascus aurantiacus. J. Ferment. Bioeng. (1998), 85(2):169-173;
Wold, F. Annu. Rev. Biochem. (1981), 50:783-814; and
U.S. Pat. No. 5,695,985.

1. An isolated catalase having an activity half-life of at least about 200 hours at a temperature of about 80° C. and a pH of about 8 and demonstrating substantially no substrate inhibition at hydrogen peroxide concentrations up to about 450 mM.

2. The isolated catalase of claim 1, wherein the catalase is derived from T. brockianus.

3. The isolated catalase of claim 1, wherein the catalase is capable of electrical communication with a biosensor.

4. An isolated thermostable catalase, wherein the catalase has a half-life of at least about 200 hours at a temperature of about 80° C. and a pH of about 8, produced by the process comprising:
growing a microorganism having catalase activity;
preparing a cell lysate;
purifying a catalase from the cell lysate.

5. The isolated thermostable catalase of claim 4, wherein growing the microorganism comprises growing T. brockianus.

6. The isolated thermostable catalase of claim 4, comprising a half-life of about 300 hours at a temperature of about 80° C. and a pH of about 8.

7. The isolated thermostable catalase of claim 4, wherein purifying the catalase comprises chromatographing the cell extract with at least one of an ion-exchange column, a hydrophobic interaction column and a gel filtration column.

8. The isolated thermostable catalase of claim 7, wherein purifying the catalase comprises chromatographing the cell extract with an ion-exchange column, a hydrophobic interaction column and a gel filtration column.

6. The isolated thermostable catalase of claim 4, wherein the catalase does not demonstrate substantial substrate inhibition of the catalase at a hydrogen peroxide concentration between about 200 and about 450 mM.

7. The isolated thermostable catalase of claim 4, further comprising immobilizing the catalase on a solid support.

8. An isolated thermostable catalase from T. brockianus, produced by the process comprising:
growing T. brockianus;
preparing a cell lysate from the microorganism;
purifying a catalase from the cell lysate.

9. The isolated thermostable catalase from T. brockianus of claim 8, comprising purifying a catalase having a half-life of at least about 200 hours at a temperature of about 80° C. and a pH of about 8.

10. The isolated thermostable catalase from T. brockianus of claim 8, the process further comprising immobilizing the catalase on a solid support to produce an immobilized catalase.

11. The isolated thermostable catalase from T. brockianus of claim 10, wherein purifying the catalase comprises chromatographing the cell extract with at least one of an ion-exchange column, a hydrophobic interaction column and a gel filtration column.

12. The isolated thermostable catalase from T. brockianus of claim 8, wherein purifying the catalase comprises chromatographing the cell extract with at least one of an ion-exchange column, a hydrophobic interaction column and a gel filtration column.

13. The isolated thermostable catalase from T. brockianus of claim 12, wherein purifying the catalase comprises chromatographing the cell extract with an ion-exchange column, a hydrophobic interaction column and a gel filtration column.

14. The isolated thermostable catalase from T. brockianus of claim 8, wherein the catalase is not substantially inhibited by hydrogen peroxide at a concentration between about 200 and about 450 mM.

15. A method of converting hydrogen peroxide to oxygen and water under conditions of high temperature and pH, the method comprising:
admixing a sample containing hydrogen peroxide and a catalase;
incubating the catalase with the hydrogen peroxide at a high temperature and at an alkaline pH; and
converting the hydrogen peroxide to oxygen and water.

16. The method according to claim 15, wherein the catalase is derived from T. brockianus.

17. The method according to claim 15, wherein incubating the catalase with the hydrogen peroxide at the high temperature comprises incubating the catalase of SEQ ID NO:5.

18. The method according to claim 15, wherein incubating the catalase with the hydrogen peroxide at the alkaline pH comprises incubating the catalase at a pH between about 8 and about 10.

19. The method according to claim 15, further comprising selecting a catalase having a half-life of about 300 hours when incubated at about 80° C. and about pH 8.

20. The method according to claim 15, further comprising obtaining the sample from bleaching of pulp, paper or textile.

21. The method according to claim 15, further comprising immobilizing the catalase on a solid support to produce an immobilized catalase.

22. The method according to claim 21, wherein admixing the sample and the catalase further comprises passing the sample through a column of the immobilized catalase, and obtaining the sample from bleaching of pulp, paper or textile.

23. The method according to claim 21, further comprising selecting a solid support having a negative charge.

24. A method of purifying a catalase, comprising:
growing a microorganism having catalase activity;
preparing a cell lysate from the microorganism;
purifying a catalase from the cell lysate by chromatography with at least one of an ion-exchange column, a hydrophobic interaction column and a gel filtration column.

25. The method according to claim 24, wherein growing the microorganism comprises growing a thermophilic microorganism.

26. The method according to claim 24, wherein growing the thermophilic microorganism comprises growing T. brockianus.

27. An isolated nucleic acid comprising a nucleic acid sequence encoding a polypeptide having the sequence set forth in SEQ ID NO:2, a polypeptide having 95% identity to the sequence set forth in SEQ ID NO:2 or a functional fragment thereof.

28. The isolated nucleic acid of claim 27, wherein the nucleic acid comprises a vector.

29. The isolated nucleic acid of claim 28, wherein the vector comprises an expression vector.

30. The isolated nucleic acid of claim 29, wherein the vector is in a host cell.

31. The isolated nucleic acid of claim 27, wherein the polypeptide comprises SEQ ID NO:5.

32. The isolated nucleic acid of claim 27, wherein the polypeptide has the sequence set forth in SEQ ID NO:2.

33. A cell, comprising the isolated nucleic acid of claim 32.

34. An isolated catalase comprising a polypeptide having the sequence set forth in SEQ ID NO:2, a polypeptide having 95% identity to the sequence set forth in SEQ ID NO:2 or a functional fragment thereof.

35. The isolated catalase of claim 34, wherein the polypeptide has the sequence set forth in SEQ ID NO:2.

36. The isolated catalase of claim 34, wherein the polypeptide comprises SEQ ID NO:5.

37. A structure for treating a process stream, comprising a catalase having an activity half-life of at least about 200 hours at a temperature of about 80° C. and a pH of about 8 and substantially no substrate inhibition at hydrogen peroxide concentrations up to about 450 mM, wherein the catalase is immobilized on a water insoluble support.

38. The structure for treating a process stream of claim 37, wherein the catalase is derived from T. brockianus.

39. The structure for treating a process stream of claim 37, wherein the water insoluble support is selected from the group consisting of cellulose, cellulose derivatives, dextran, agarose, carboxymethylcellulose and chitonsan.

40. The structure for treating a process stream of claim 39, wherein the water insoluble support comprises carboxymethylcellulose.

41. The structure for treating a process stream of claim 37, wherein the catalase comprises SEQ ID NO:5.
